(19) United States
(12) Patent Application Publication
(10) Pub. No.: US 2026/0324951 A1
(43) Pub. Date: Oct. 22, 2026

(54) IMAGING DEVICE AND MEDICAL OBSERVATION SYSTEM

(71) Applicant: SONY GROUP CORPORATION, TOKYO (JP)

(72) Inventors: SHINYA KUNIMATSU, TOKYO (JP); TOMOYUKI OKI, TOKYO (JP)

(21) Appl. No.: 19/110,426

(22) PCT Filed: Sep. 4, 2023
(86) PCT No.: PCT/JP2023/032205

§ 371 Date: Mar. 11, 2025

(30) Foreign Application Priority Data
Sep. 16, 2022 (JP) 2022-148083

(51) Int. Cl. A61B 1/00; A61B 1/04; A61B 1/06; A61B 1/313

(52) U.S. Cl. CPC A61B 1/00096; A61B 1/00009; A61B 1/043; A61B 1/0653; A61B 1/3132

(57) ABSTRACT

An imaging device includes: a prism configured to separate visible light and special light from each other, the visible light and the special light included in light from an observation target; a visible light imaging sensor configured to image the visible light separated by the prism; and a special light imaging sensor configured to image the special light separated by the prism. A ratio of spectral transmittance of the visible light toward the special light imaging sensor to spectral transmittance of the visible light toward the visible light imaging sensor in the prism is 0.25 or more and 4 or less, and spectral transmittance of the special light toward the special light imaging sensor in the prism is greater than 90%.

### FIELD

The present disclosure relates to an imaging device and a medical observation system.

### BACKGROUND

For example, Patent Literature 1 discloses a technique of separating visible light and special light from an observation target and capturing individual light images with different imaging sensors.

### CITATION LIST

### Patent Literature

Patent Literature 1: JP 2017-53890 A

### SUMMARY

### Technical Problem

The light from the observation target may include not only special light outside the visible light wavelength band but also visible special light belonging to the visible light band. When visible light (normal visible light), which is normal light rather than special light, and visible special light, are imaged by an identical visible light imaging sensor, it is difficult to image the visible special light with high clarity because of the reason that the visible special light is weaker than the normal visible light, for example.

One aspect of the present disclosure enables imaging of visible special light with high clarity even when observing both normal visible light and visible special light.

### Solution to Problem

An imaging device according to one aspect of the present disclosure includes: a prism configured to separate visible light and special light from each other, the visible light and the special light included in light from an observation target; a visible light imaging sensor configured to image the visible light separated by the prism; and a special light imaging sensor configured to image the special light separated by the prism, wherein a ratio of spectral transmittance of the visible light toward the special light imaging sensor to spectral transmittance of the visible light toward the visible light imaging sensor in the prism is 0.25 or more and 4 or less, and spectral transmittance of the special light toward the special light imaging sensor in the prism is greater than 90%.

An imaging device according to one aspect of the present disclosure includes: a prism configured to separate visible light and special light from each other, the visible light and the special light included in light from an observation target; a visible light imaging sensor configured to image the visible light separated by the prism; a special light imaging sensor configured to image the special light separated by the prism; and a visible special light imaging sensor configured to image visible special light included in the visible light separated by the prism, wherein a ratio of spectral transmittance of the visible light toward the visible special light imaging sensor to spectral transmittance of the visible light toward the visible light imaging sensor in the prism is greater than 0.2 and less than 5, and spectral transmittance of the special light toward the special light imaging sensor in the prism is greater than 90%.

A medical observation system according to one aspect of the present disclosure includes an imaging device configured to image a surgical site, wherein the imaging device includes: a prism configured to separate visible light and special light from each other, the visible light and the special light included in light from the surgical site; a visible light imaging sensor configured to image the visible light separated by the prism; and a special light imaging sensor configured to image the special light separated by the prism, a ratio of spectral transmittance of the visible light toward the special light imaging sensor to spectral transmittance of the visible light toward the visible light imaging sensor in the prism is 0.25 or more and 4 or less, and spectral transmittance of the special light toward the special light imaging sensor from the prism is greater than 90%.

### BRIEF DESCRIPTION OF DRAWINGS

FIG. 1 is a diagram illustrating an example of a schematic configuration of an imaging device according to a first embodiment.

FIG. 2 is a diagram illustrating an example of characteristics of a dichroic film 65.

FIG. 3 is a diagram illustrating an example of spectral transmittance of a prism.

FIG. 4 is a diagram illustrating an example of spectral transmittance of a prism toward a special light imaging sensor 9.

FIG. 5 is a diagram illustrating an example of a schematic configuration of a visible light imaging sensor.

FIG. 6 is a diagram illustrating an example of a pixel interval of the visible light imaging sensor.

FIG. 7 is a diagram illustrating an example of a pixel interval of the visible light imaging sensor.

FIG. 8 is a diagram illustrating an example of characteristics of a band pass filter.

FIG. 9 is a diagram illustrating an example of a schematic configuration of a special light imaging sensor.

FIG. 10 is a diagram illustrating an example of a pixel interval of the special light imaging sensor.

FIG. 11 is a diagram illustrating an example of a pixel interval of the special light imaging sensor.

FIG. 12 is a diagram illustrating an example of a holding member.

FIG. 13 is a diagram illustrating an example of a schematic configuration of an imaging device according to a second embodiment.

FIG. 14 is a diagram illustrating an example of characteristics of a dichroic film 66.

FIG. 15 is a diagram illustrating an example of spectral transmittance of a prism.

FIG. 16 is a diagram illustrating an example of a schematic configuration of an endoscope system.

FIG. 17 is a block diagram illustrating an example of a functional configuration of a camera and a camera control unit (CCU) illustrated in FIG. 16.

FIG. 18 is a diagram illustrating an example of a schematic configuration of a microscopic surgery system.

### DESCRIPTION OF EMBODIMENTS

Embodiments of the present disclosure will be described below in detail with reference to the drawings. Note that, in each of the following embodiments, the same elements are denoted by the same reference symbols, and a repetitive description thereof will be omitted.

The present disclosure will be described in the following order of items.
0. Introduction
1. First Embodiment
2. Second Embodiment
3. Modifications
4. Application example
5. Exemplary effects

### 0. Introduction

For example, a device such as a medical endoscope uses a wavelength separation prism (two-plate prism configuration) in order to separate light into visible light and special light (for example, near-infrared special light) and allow the individual beams of light to be received by corresponding imaging sensors to achieve imaging. In order to achieve separation into individual beams of light, a dichroic film applied on a bonding surface between the blocks (glass members) of the prism is used. A conventionally used film is a dichroic film that reflects 100% of one beam out of visible light and special light beams and transmits 100% of the other light beam. That is, for example, spectral characteristics (ratio of reflection and transmission) for visible light and special light are 10:0 and 0:10.

The light from the observation target sometimes includes visible special light belonging to the visible light band. Visible light that is normal and is not visible special light is referred to as normal visible light. In the above-described two-plate prism configuration, normal visible light and visible special light are imaged by the same visible light imaging sensor, while special light is imaged by the special light imaging sensor. The special light can be imaged with high sensitivity by a device such as a monochrome HD imaging sensor, for example. However, the visible special light is imaged in time division with normal light by RGB Bayer imaging sensors, leading to lower light receiving sensitivity and degradation of responsiveness.

At least some of the problems as described above can be handled by the disclosed technology. For example, the spectral characteristic of the dichroic film of the prism for visible light is designed to be 5:5 (or may be 6:4, 7:3, 8:2, etc.) instead of 10:0. The spectral characteristic of the special light may be designed to be 0:10. The relationship between reflection and transmission may be reversed depending on the arrangement of the imaging sensor. The visible special light may be, for example, fluorescence of an agent, and the visible light band in that case may be only the fluorescence wavelength band of the agent. An example of a fluorescence wavelength band of an agent is about 500 nm to 550 nm. The visible light and the special light may be subjected to wavelength separation by a prism, and may be simultaneously received by a plurality of imaging sensors having mutually different wavelength band sensitivities (multicolor or monochrome), cell sizes, and/or imaging sizes. The special light is, for example, near-infrared light lower in energy than visible light, and such light can be imaged with high clarity. It is allowable to dispose a depolarizer on the front side of the prism to cancel the polarization dependence of the light from the imaging target (subject). The imaging device may be used in combination with a device such as a rigid scope and a microscope, for example.

### 1. First Embodiment

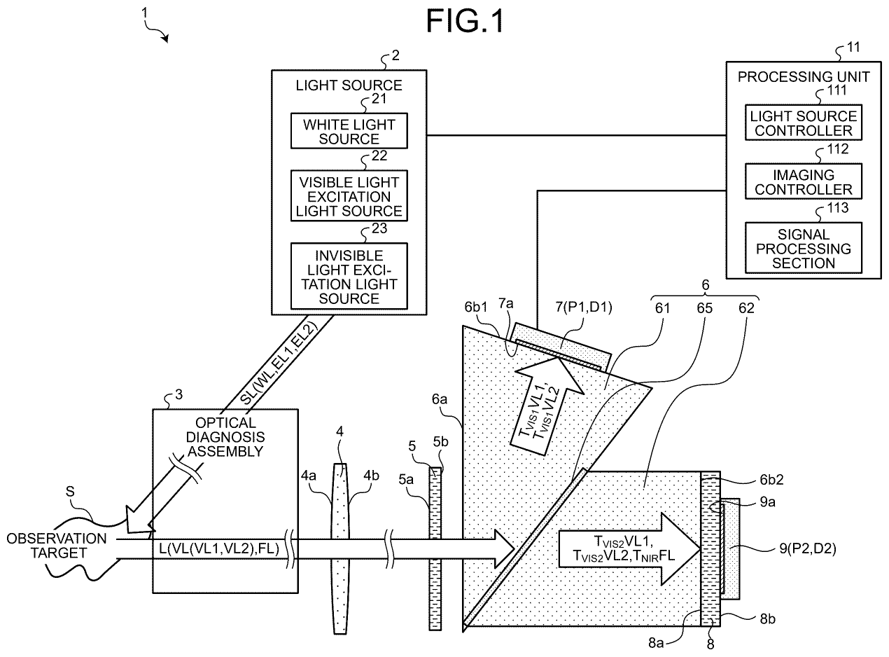

FIG. 1 is a diagram illustrating an example of a schematic configuration of an imaging device according to a first embodiment. An imaging device 1 images an observation target S (subject). FIG. 1 schematically illustrates each element of the observation target S and the imaging device 1 in side view. The imaging device 1 images light from the observation target S. Note that “imaging” may be understood to include “shooting”, and “image” may be understood to include “video”. Imaging and images may be appropriately rephrased as shooting and video respectively within a range without contradiction.

The observation of the observation target S includes visible light observation and special light observation. Accordingly, imaging of the observation target S by the imaging device 1 includes imaging of visible light and imaging of special light. Unless otherwise specified, the term special light denotes light (for example, infrared light and near-infrared light) belonging to a wavelength band different from the visible light wavelength band. In the present embodiment, besides the special light, there is also visible special light belonging to the visible light wavelength band. The observation of the observation target S also includes observation of visible special light.

The imaging device 1 includes a light source 2, an optical diagnosis assembly 3, an imaging lens 4, an optical element 5, a prism 6, a visible light imaging sensor 7, a band pass filter 8, a special light imaging sensor 9, and a processing unit 11. In addition, some beams of light are schematically illustrated by outlined arrows.

The light source 2 outputs light SL. The light SL includes white light WL, excitation light EL1, and excitation light EL2. The excitation light EL1 is visible light excitation light and may be laser light. The excitation light EL2 is invisible light excitation light. The excitation light EL2 is near-infrared light, for example, and in this case, the excitation light EL2 may be laser light having a wavelength of about 780±30 nm. In the example illustrated in FIG. 1, the light source 2 includes a white light source 21, a visible light excitation light source 22, and an invisible light excitation light source 23. The white light source 21 outputs the white light WL. The white light source 21 may include a Light Emitting Diode (LED) that emits white light, or may include an LED that emits red light, an LED that emits green light, and an LED that emits blue light (RGB-LED). Alternatively, the white light source 21 may include a lamp such as a halogen lamp. The visible light excitation light source 22 outputs the excitation light EL1. The invisible light excitation light source 23 outputs the excitation light EL2.

The optical diagnosis assembly 3 guides the light SL from the light source 2, specifically, the white light WL from the white light source 21, the excitation light EL1 from the visible light excitation light source 22, and the excitation light EL2 from the invisible light excitation light source 23, to the observation target S. The optical diagnosis assembly 3 can constitute a part of an imaging system (medical observation system) including devices such as a laparoscope, a rigid endoscope, a flexible endoscope, a camera, and a surgical microscope, for example. The optical diagnosis assembly 3 may include an optical system for light transmission, and a housing that accommodates the optical system, for example. The light SL from the light source 2 is emitted to the observation target S via the optical diagnosis assembly 3.

The observation target S is illuminated by being irradiated with the white light WL included in the light SL. In addition, the observation target S is provided with (by injection, for example) an agent excited by being irradiated with the excitation light EL1 and the excitation light EL2 included in the light SL. Due to these illumination and excitation, light L including the visible light VL and the special light FL is generated on the observation target S. The visible light VL includes normal visible light VL1 and visible special light VL2. The normal visible light VL1 includes light reflected by the observation target S irradiated with the white light WL. The visible special light VL2 includes light (for example, fluorescence) emitted by an agent injected into the observation target S and excited by the excitation light EL1. The special light FL includes light (for example, fluorescence) emitted by the agent excited by the excitation light EL2. For example, two types of agents, namely, an agent that emits the visible special light VL2 and an agent that emits the special light FL, are injected into the observation target S.

The optical diagnosis assembly 3 guides the light L from the observation target S, that is, the visible light VL and the special light FL, to the prism 6, more specifically, to the imaging lens 4 provided on the front side (observation target S side) of the prism 6 in this example. The light L from the observation target S is incident on the imaging lens 4 via the optical diagnosis assembly 3.

The imaging lens 4 is provided between the observation target S and the prism 6, more specifically, between the optical diagnosis assembly 3 and the optical element 5 in this example. The imaging lens 4 includes one or more condensing lenses, for example. An incoming surface of the imaging lens 4, specifically, an incoming surface of the light L from the observation target S, is denoted as an incoming surface 4a in the drawing. The outgoing surface of the light L is denoted as an outgoing surface 4b in the drawing. The light L from the outgoing surface 4b of the imaging lens 4 is incident on the optical element 5.

The optical element 5 is provided between the observation target S and the prism 6, more specifically, between the imaging lens 4 and the prism 6 in this example. In the optical element 5, a surface on which the light L is incident is denoted as an incoming surface 5a in the drawing. A surface from which the light L is emitted is denoted as an outgoing surface 5b in the drawing. The light L from the outgoing surface 5b is incident on the prism 6. The incoming surface 5a and the outgoing surface 5b may be surfaces perpendicular to the optical axis.

An example of the optical element 5 is a depolarizer. In a case where the light L is polarized light including specific polarized light, the optical element 5 cancels the polarized light. For example, non-polarized light (unpolarized light) in which various types of polarized light are mixed is created. An example of the polarized light is polarized light including P-polarized light and S-polarized light. The optical element 5 cancels the polarization of the light L to form unpolarized light, and allows the obtained unpolarized light to be incident on the prism 6.

Another example of the optical element 5 is an excitation light cut filter. In a case where the excitation light EL1 and the excitation light EL2 are included in the light L, the optical element 5 reflects or attenuates the excitation light EL1 and the excitation light EL2 and passes other light. Note that, to the extent that there is no contradiction, “transmitting” may be understood as meaning of “passing”, and these may be rephrased as appropriate.

Both the depolarizer and the excitation light cut filter described above may be included in the optical element 5.

The prism 6 separates and outputs the light L from the observation target S, more specifically, the visible light VL and the special light FL included in the light L after transmitting through the imaging lens 4 and the optical element 5 in this example. The prism 6 includes a block 61, a block 62, and a dichroic film 65. A surface of the prism 6, that is, a surface on which the light L is incident is denoted as an incoming surface 6a in the drawing. The surface from which the visible light VL is emitted is denoted as an outgoing surface 6b1 in the drawing. A surface from which the special light FL is emitted is denoted as an outgoing surface 6b2 in the drawing. The outgoing surface 6b1 is a part of the surfaces of the block 61. The outgoing surface 6b2 is a part of the surfaces of the block 62.

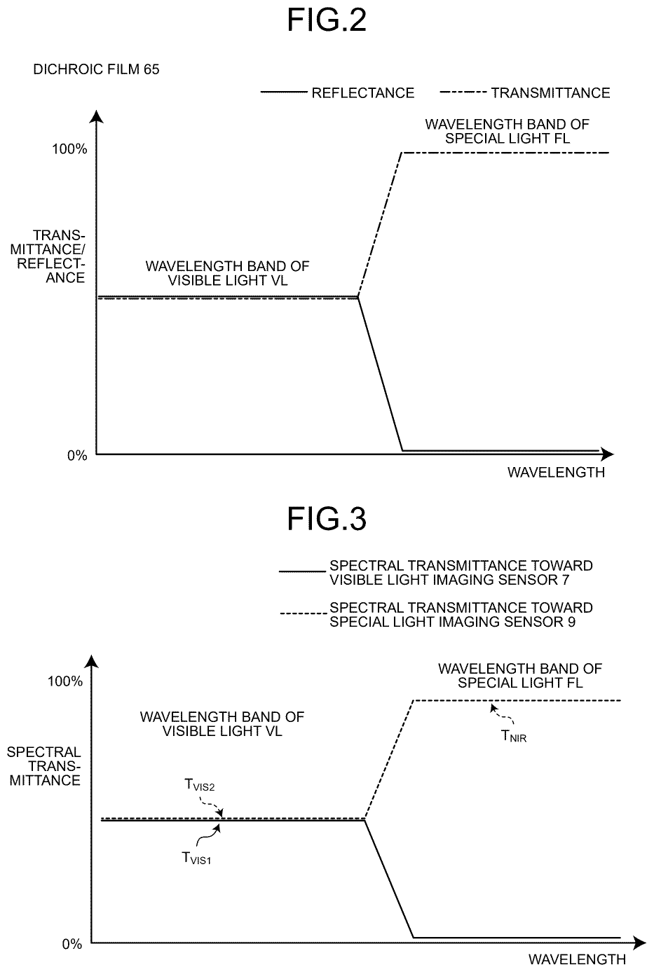

The dichroic film 65 is provided between the block 61 and the block 62 so that the light L from the incoming surface 6a of the prism 6 is incident. In this example, the dichroic film 65 is deposited (formed) on the block 61. The dichroic film 65 reflects a part of the visible light VL and transmits the special light FL and the rest of the visible light VL. This will be described with reference to FIG. 2.

FIG. 2 is a diagram illustrating an example of characteristics of the dichroic film 65. The horizontal axis of the graph indicates the wavelength. The vertical axis of the graph represents transmittance and reflectance (%). The transmittance and the reflectance in the wavelength band of the visible light VL are values apart from 0% and 100%. In this example, both the reflectance and the transmittance in the wavelength band of the visible light VL are values exactly or close to 50%. The transmittance of the special light FL in the wavelength band is a value exactly or close to 100%. The dichroic film 65 is designed to impart spectral transmittance characteristics of the prism 6 described below.

Returning to FIG. 1, the block 61 of the prism 6 is a prism block (first block) that guides the visible light VL to the visible light imaging sensor 7. The block 61 emits the light reflected by the dichroic film 65 from the outgoing surface 6b1. The block 62 is a prism block (second block) that guides the visible light VL and the special light FL to the special light imaging sensor 9. The block 62 emits the light transmitted through the dichroic film 65 from the outgoing surface 6b2.

The spectral transmittance of the visible light VL to the visible light imaging sensor 7 in the prism 6 is denoted as spectral transmittance T_{VIS1}. The spectral transmittance of the visible light VL to the special light imaging sensor 9 in the prism 6 is denoted as spectral transmittance T_{VIS2}. The spectral transmittance from the prism 6 toward the special light imaging sensor 9 is denoted as spectral transmittance T_{NIR}. This will be described with reference to FIG. 3.

FIG. 3 is a diagram illustrating an example of spectral transmittance of a prism. The horizontal axis of the graph indicates the wavelength. The vertical axis of the graph represents spectral transmittance (%). The spectral transmittance toward the visible light imaging sensor 7 in the wavelength band of the visible light VL is the spectral transmittance T_{VIS1}. The spectral transmittance toward the special light imaging sensor 9 in the wavelength band of the visible light VL is the spectral transmittance T_{VIS2}. The spectral transmittance toward the special light imaging sensor 9 in the wavelength band of the special light FL is the spectral transmittance T_{NIR}. The spectral transmittance T_{VIS1} and the spectral transmittance T_{VIS2} are values apart from 0% and 100%. The spectral transmittance T_{NIR} may be a value exactly or close to 100%. More specific design of the spectral transmittance T_{VIS1}, the spectral transmittance T_{VIS2}, and the spectral transmittance T_{NIR} will be described below again.

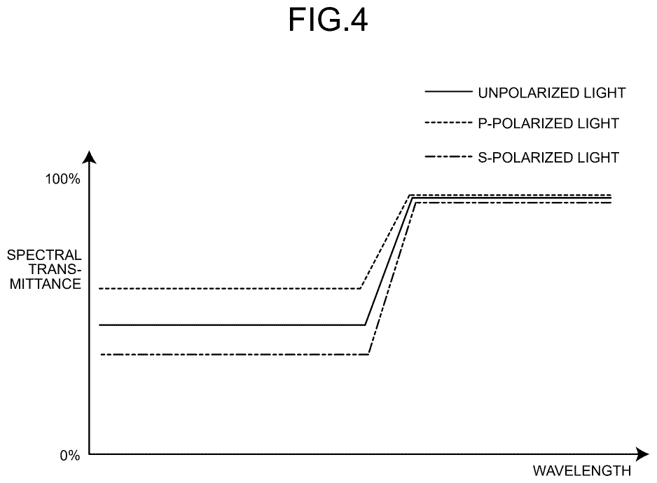

Note that the spectral transmittance of the prism 6 may depend on polarization. This will be described with reference to FIG. 4.

FIG. 4 is a diagram illustrating an example of spectral transmittance of a prism toward a special light imaging sensor 9. The illustrates the spectral transmittance of the unpolarized light, the spectral change transmittance of the P-polarized light, and the spectral transmittance of the S-polarized light. The P-polarized light and the S-polarized light have different spectral transmittances. When the light L incident on the prism 6 includes the P-polarized light and the S-polarized light, there will be fluctuation in the light quantities of the visible light VL and the special light FL separated and output by the prism 6. As described above, by providing the optical element 5 including the depolarizer on the front side (observation target S side) of the prism 6, the light L incident on the prism 6 can be turned into unpolarized light. This makes it possible to suppress the above-described light quantity fluctuation and to image the visible light VL and the special light FL with stable exposure.

Returning to FIG. 1, the normal visible light VL1 emitted from the outgoing surface 6b1 of the prism 6 has a light quantity (T_{VIS1}VL1) obtained by multiplying the light quantity of the normal visible light VL1 incident on the incoming surface 6a by the spectral transmittance T_{VIS1}. Similarly, the visible special light VL2 emitted has a light quantity (T_{VIS1}VL2) obtained by multiplying the light quantity of the visible special light VL2 incident on the incoming surface 6a by the spectral transmittance T_{VIS1}.

The normal visible light VL1 emitted from the outgoing surface 6b2 of the prism 6 has a light quantity (T_{VIS2}VL1) obtained by multiplying the light quantity of the visible special light VL2 incident on the incoming surface 6a by the spectral transmittance T_{VIS2}. Similarly, the visible special light VL2 emitted has a light quantity (T_{VIS2}VL2) obtained by multiplying the light quantity of the visible special light VL2 incident on the incoming surface 6a by the spectral transmittance T_{VIS2}. Similarly, the special light FL emitted has a light quantity (T_{NIR}FL) obtained by multiplying the light quantity of the special light FL incident on the incoming surface 6a by the spectral transmittance T_{NIR}.

The visible light VL (normal visible light VL1 and visible special light VL2) emitted from the outgoing surface 6b1 of the prism 6 forms an image, which is captured by the visible light imaging sensor 7. The visible light imaging sensor 7 is directly or indirectly fixed to the prism 6 so as to be located at an image forming position of the visible light VL.

The visible light imaging sensor 7 images the visible light VL from the prism 6. The visible light imaging sensor 7 includes a light receiving surface 7a. The visible light imaging sensor 7 is provided such that the light receiving surface 7a faces the outgoing surface 6b1 of the prism 6. In this example, the visible light imaging sensor 7 is provided (bonded, for example) on the outgoing surface 6b1 of the block 61 of the prism 6. The visible light imaging sensor 7 is, for example, a Complementary Metal Oxide Semiconductor (CMOS) image sensor designed to image visible light VL (normal visible light VL1 and visible special light VL2). The normal visible light VL1 and the visible special light VL2 may be imaged in time division, and details thereof will be described below.

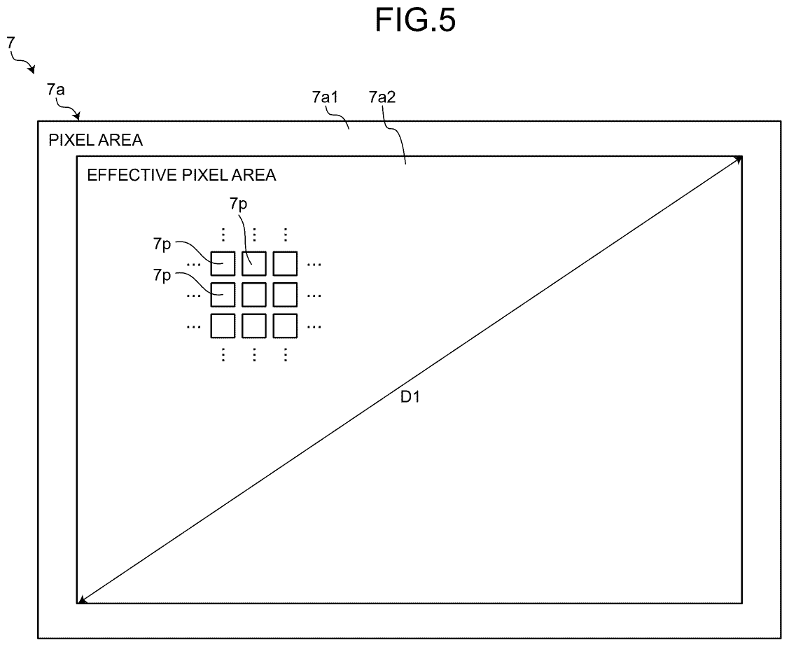

In the first embodiment, the pixel interval (pixel pitch) of the visible light imaging sensor 7 is denoted as a pixel interval P1. An effective pixel area diagonal length of the visible light imaging sensor 7 is denoted as an effective pixel area diagonal length D1. This will be further described with reference to FIGS. 5 to 7.

FIG. 5 is a diagram illustrating an example of a schematic configuration of the visible light imaging sensor. The figure schematically illustrates the visible light imaging sensor 7 in front view. The visible light imaging sensor 7 includes a plurality of pixels 7p. Each pixel 7p is designed to efficiently receive visible light. For example, the plurality of pixels 7p includes a pixel that receives blue light, a pixel that receives red light, and a pixel that receives green light. Each pixel 7p includes: a filter that transmits light of a corresponding color; a photoelectric conversion sensor that receives light transmitted through the filter and generates a charge according to the quantity of received light; and a transistor (pixel circuit, for example) that controls light reception by the photoelectric conversion sensor and extracts an electric signal according to the charge. The pixel array may include a Bayer array, for example, but is not limited to this array pattern, and may be implemented by adopting various known pixel array patterns.

The visible light imaging sensor 7 includes a pixel area 7a1 and an effective pixel area 7a2. The effective pixel area 7a2 is an area inside the pixel area 7a1 and is smaller than the pixel area 7a1. The plurality of pixels 7p is arranged in an array pattern over the entire pixel area 7a1 so as to define the light receiving surface 7a. Among these pixels 7p, the pixel 7p arranged in the effective pixel area 7a2 is an effective pixel to be actually used for imaging. The diagonal length of the effective pixel area 7a2 is to be the effective pixel area diagonal length D1. The larger the effective pixel area diagonal length D1, the larger the effective pixel area 7a2. In this sense, the effective pixel area diagonal length D1 may be appropriately rephrased as the effective pixel area 7a2, or may be rephrased as the term indicating the size of the visible light imaging sensor 7.

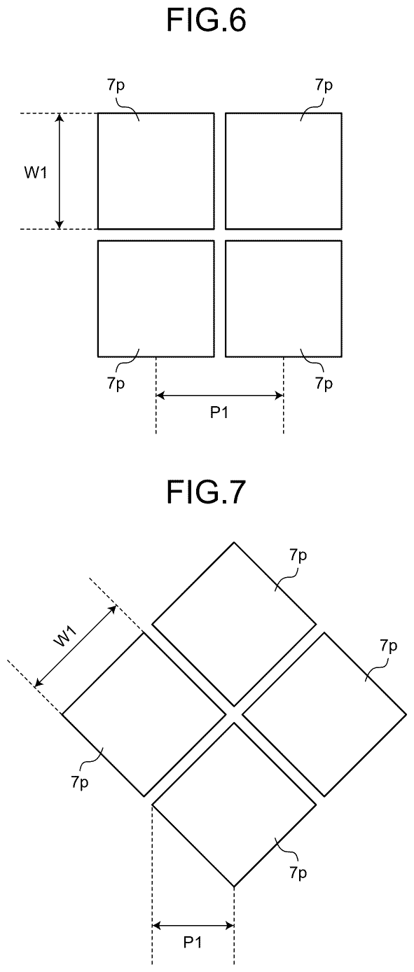

FIGS. 6 and 7 are diagrams illustrating examples of pixel intervals of the visible light imaging sensor. For ease of understanding, only four pixels 7p are illustrated. The pixels 7p may be arranged side by side in the horizontal direction and the vertical direction as illustrated in FIG. 6, or may be arranged side by side in a diagonal direction (a direction between the vertical direction and the horizontal direction) as illustrated in FIG. 7. In this example, each pixel 7p has a square shape, and the length of one side thereof defines the size of the pixel 7p. The size of the pixel 7p is denoted as a pixel size W1 in the drawing. The distance between the centers of the adjacent pixels 7p is the pixel interval P1. The larger the pixel interval P1, the larger the pixel size W1. In this sense, the pixel interval P1 may be appropriately rephrased as the pixel size W1.

When the effective pixel area diagonal length D1 is the same (the size of the visible light imaging sensor 7 is the same), the shorter the pixel interval P1, the higher the resolution. The larger the pixel interval P1, the lower the resolution.

Returning to FIG. 1, the visible light VL (normal visible light VL1 and visible special light VL2) and the special light FL from the outgoing surface 6b2 of the prism 6 are incident on the band pass filter 8.

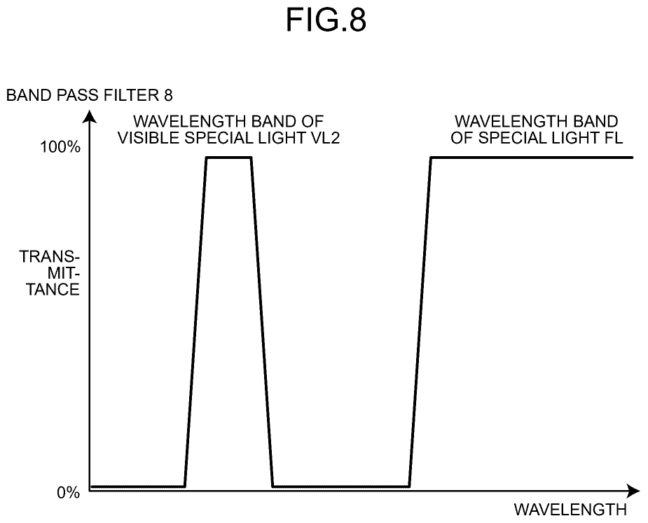

The band pass filter 8 is provided between the block 62 of the prism 6 and the special light imaging sensor 9. The band pass filter 8 transmits the visible special light VL2 and the special light FL, and reflects or attenuates the normal visible light VL1 belonging to a wavelength band different from the wavelength band of the visible special light VL2. There is a possibility that the unwanted normal visible light VL1 causes flare which leads to a failure in obtaining a clear image of the special light FL. However, the presence of the band pass filter 8 suppresses incidence of such unwanted normal visible light VL1 on the special light imaging sensor 9. This makes it possible to obtain a high-quality image with a high S/N ratio. In the band pass filter 8, a surface on which the normal visible light VL1, the visible special light VL2, and the special light FL are incident is denoted as an incoming surface 8a in the drawing. A surface from which the visible special light VL2 and the special light FL are emitted is denoted as an outgoing surface 8b in the drawing. The band pass filter 8 will be described with reference to FIG. 8.

FIG. 8 is a diagram illustrating an example of characteristics of the band pass filter. The transmittance is high (reflection or attenuation level is low) in the wavelength band of the visible special light VL2 and the wavelength band of the special light FL, and the transmittance is low (reflection or attenuation is high) in other wavelength bands. For example, the band pass filter 8 may transmit 90% or more of light having a wavelength of at least 750 nm to 850 nm and attenuate light having a wavelength of 400 nm to 600 nm (excluding the wavelength of the visible special light VL2) to a level 10% or less. More specifically, the band pass filter 8 may transmit 90% or more of light having a wavelength of at least 820 n to 850 nm and reflect or attenuate light having a wavelength of 400 nm to 600 nm (excluding the wavelength of the visible special light VL2) to a level of 10% or less. The optical density of the visible light VL (excluding the visible special light VL2) emitted from the outgoing surface 8b of the band pass filter 8 with respect to the visible light VL (excluding the visible special light VL2) incident on the incoming surface 6a of the prism 6 may be OD3 or more.

Returning to FIG. 1, the incoming surface 8a of the band pass filter 8 may be a surface perpendicular to the optical axis. The band pass filter 8 is provided for the prism 6 such that the incoming surface 8a faces the outgoing surface 6b2 of the block 62 of the prism 6. In the example illustrated in FIG. 1, the band pass filter 8 is provided (bonded, for example) on the outgoing surface 6b2 of the block 62 of the prism 6.

The visible special light VL2 and the special light FL from the outgoing surface 8b of the band pass filter 8 form an image, which is captured by the special light imaging sensor 9. The special light imaging sensor 9 is directly or indirectly fixed to the prism 6 so as to be located at one image forming position of the visible special light VL2 and the special light FL, for example, at the image forming position of the special light FL.

The special light imaging sensor 9 images the visible special light VL2 and the special light FL from the prism 6. The special light imaging sensor 9 includes a light receiving surface 9a. The special light imaging sensor 9 is provided such that the light receiving surface 9a faces the outgoing surface 6b2 of the prism 6. In this example, the special light imaging sensor 9 is provided (bonded, for example) on the outgoing surface 8b of the band pass filter 8 so as to face the outgoing surface 8b of the band pass filter 8. The special light imaging sensor 9 is, for example, a Complementary Metal Oxide Semiconductor (CMOS) image sensor designed to image the visible special light VL2 and the special light FL.

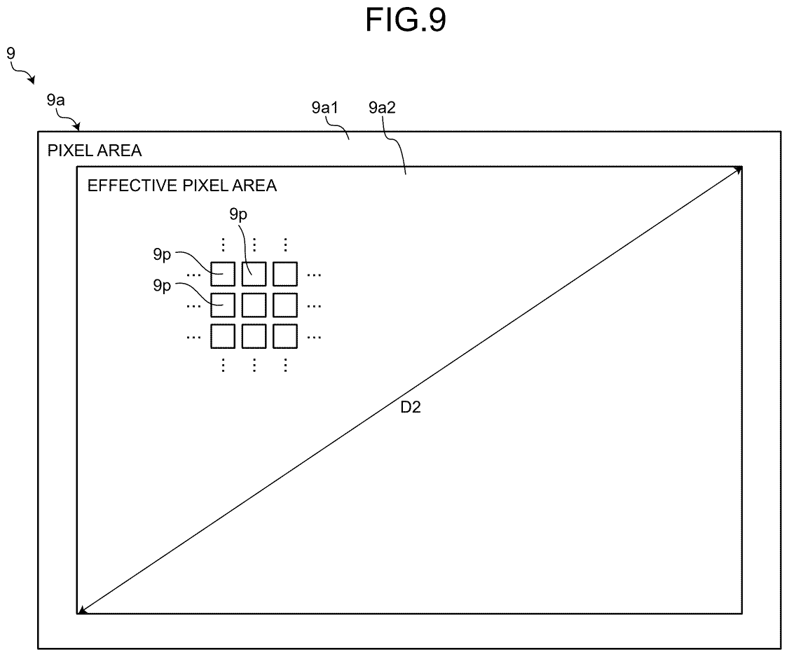

The pixel interval of the special light imaging sensor 9 is denoted as a pixel interval P2. An effective pixel area diagonal length of the special light imaging sensor 9 is denoted as an effective pixel area diagonal length D2. This will be further described with reference to FIGS. 9 to 11.

FIG. 9 is a diagram illustrating an example of a schematic configuration of a special light imaging sensor. The figure schematically illustrates the special light imaging sensor 9 in front view. The special light imaging sensor 9 includes a plurality of pixels 9p. Each pixel 9p is designed to efficiently receive the visible special light VL2 and the special light FL (for example, the special light FL in particular). For example, each pixel 9p includes: a filter that transmits the visible special light VL2 and the special light FL; a photoelectric conversion sensor that receives light transmitted through the filter and generates a charge according to the quantity of received light; and a transistor (pixel circuit, for example) that controls light reception by the photoelectric conversion sensor and extracts an electric signal according to the charge. The pixel array may be determined by adopting various known pixel arrays.

The special light imaging sensor 9 includes a pixel area 9a1 and an effective pixel area 9a2. The plurality of pixels 9p is arranged in an array pattern over the entire pixel area 9a 1 so as to define the light receiving surface 9a. Among these pixels 9p, the pixel 9p arranged in the effective pixel area 9a2 is an effective pixel to be actually used for imaging. The diagonal length of the effective pixel area 9a2 is to be the effective pixel area diagonal length D2. When the effective pixel area diagonal length D2 is the same (the size of the special light imaging sensor 9 is the same), the shorter the pixel interval P2, the higher the resolution. The larger the pixel interval P2, the lower the resolution.

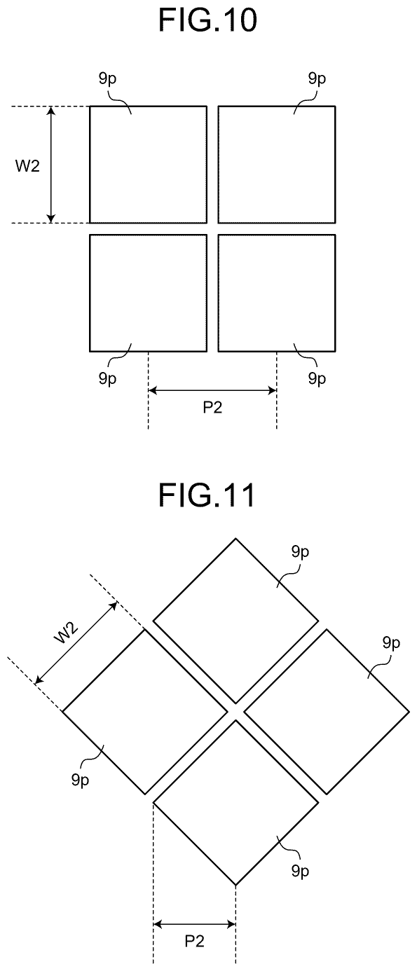

FIGS. 10 and 11 are diagrams illustrating examples of pixel intervals of the special light imaging sensor. The pixels 9p may be arranged side by side in the horizontal direction and the vertical direction as illustrated in FIG. 10, or may be arranged side by side in a diagonal direction as illustrated in FIG. 11. The size of the pixel 9p is denoted as a pixel size W2 in the drawing. The distance between the centers of the adjacent pixels 9p is the pixel interval P2. The larger the pixel size W2, the larger the pixel interval P2.

Returning to FIG. 1, the processing unit 11 performs various types of information processing in the imaging device 1. The processing unit 11 includes devices (not illustrated) such as a processor and a storage device, for example, and is designed to implement functions of the processing unit 11 described below. The design of the unit may be provided as a software design or a hardware design. In FIG. 1, some functions of the processing unit 11 are indicated by functional blocks. In this example, the processing unit 11 includes a light source controller 111, an imaging controller 112, and a signal processing section 113.

The light source controller 111 controls emission of the light SL by the light source 2. The control by the light source controller 111 includes time-division irradiation control. The processing unit 11 controls the light source 2 so that the light source 2 irradiates the observation target S with the white light WL and the excitation light EL1 at different timings.

The imaging controller 112 controls the timing of reception of the imaging signal from the visible light imaging sensor 7. The imaging signal includes, for example, a pixel signal from each pixel 7p of the visible light imaging sensor 7. The pixel signal may be an electric signal (such as a voltage signal) corresponding to the quantity of received light of the corresponding pixel 7p. The imaging controller 112 receives an imaging signal when the observation target S is irradiated with the white light WL as a normal visible light image signal, and receives an imaging signal when the observation target S is irradiated with the excitation light EL1 as a visible special light image signal. The reception of these signals is performed in synchronization with the control of the light source 2 by the light source controller 111.

The signal processing section 113 processes the imaging signal from the visible light imaging sensor 7. An example of processing of the imaging signal by the signal processing section 113 is generation of an image (image data) based on the imaging signal. More specifically, the signal processing section 113 generates an image based on the normal visible light image signal described above. With this operation, an image for normal observation is obtained. Furthermore, the signal processing section 113 generates an image based on the above-described visible special light image signal. With this operation, an image for visible special light observation is obtained.

In the imaging device 1 described above, the visible light VL (the normal visible light VL1 and the visible special light VL2) and the special light FL from the observation target S are separated from each other by the prism 6, and these beams of light are imaged by the visible light imaging sensor 7 and the special light imaging sensor 9, thereby performing visible light observation and special light observation of the observation target S. The normal visible light VL1 and the visible special light VL2 are imaged in time division by the visible light imaging sensor 7. This alone might not be sufficient to achieve clear imaging of the visible special light VL2. Furthermore, regarding the special light FL, due to a reason that the special light FL is weaker than the normal visible light VL1, for example, the sensitivity of the special light imaging sensor 9 might be insufficient, leading to a failure in special light observation with high clarity. The imaging device 1 according to the first embodiment is designed to be able to image the visible special light VL2 with high clarity even when observing both the normal visible light VL1 and the visible special light VL2. In addition, the imaging device 1 may be designed so to achieve imaging of the normal visible light VL1 with high resolution and achieve imaging of the special light FL with high clarity as well. Several design conditions will be described. Among the design conditions described below, non-exclusive design conditions may be flexibly combined.

### <Design Condition 1-1>

In one embodiment, a ratio (T_{VIS2}/T_{VIS1}) of the spectral transmittance T_{VIS2} of the visible light VL toward the special light imaging sensor 9 to the spectral transmittance T_{VIS1} of the visible light VL toward the visible light imaging sensor 7 in the prism 6 may be 0.25 or more and 4 or less. The spectral transmittance T_{NIR} of the special light FL for the special light imaging sensor 9 in the prism 6 may be greater than 90%. That is, the dichroic film 65 of the prism 6 may be designed so as to satisfy the following conditional expression (1).

$$0.25 \leq \frac{{T}_{V I S 2}}{{T}_{V I S 1}} \leq 4 and {T}_{NIR} > 90 \% \qquad ( 1 )$$

For example, the spectral transmittance T_{VIS1}, the spectral transmittance T_{VIS2}, and the spectral transmittance T_{NIR} may be designed such that 50% of the visible light VL (the normal visible light VL1 and the visible special light VL2) is to be received by each of the visible light imaging sensor 7 and the special light imaging sensor 9, and the special light FL is to be received by the special light imaging sensor 9. When the visible special light VL2 is weak, the ratio (T_{VIS2}/T_{VIS1}) may preferably be brought close to the upper limit value of the conditional expression (1). In the case of using the upper limit value, 20% of the visible light VL is to be received by the visible light imaging sensor 7, and 80% of the visible light VL is to be received by the special light imaging sensor 9. This makes it possible to image the visible special light VL2 with high clarity while imaging the normal visible light VL1. When the light quantity of the visible special light VL2 is sufficient, the ratio (T_{VIS2}/T_{VIS1}) may preferably be brought close to the lower limit value of the conditional expression (1). In the case of using the lower limit value, 80% of the visible light VL is to be received by the visible light imaging sensor 7, and 20% of the visible light VL is to be received by the special light imaging sensor 9. A clear visible light image can be obtained, and the special light imaging sensor 9 with high sensitivity can image visible special light with high sensitivity.

Incidentally, the lower limit value of the ratio (T_{VIS2}/T_{VIS1}) may be greater than the value illustrated in the above conditional expression (1). Examples of such lower limit values include 0.5, 0.75, 1.0, 1.25, 1.5, 1.75, and 2.0. The upper limit value may be smaller than the value illustrated in the conditional expression (1). Examples of such upper limit values include 3.75, 3.5, 3.25, 3.0, 2.75, 2.5, and 2.25.

### <Design Condition 1-2>

In one embodiment, the ratio (P2/P1) of the pixel interval P2 of the special light imaging sensor 9 to the pixel interval P1 of the visible light imaging sensor 7 may be 1 or more and less than 5. That is, the pixel interval P1 of the visible light imaging sensor 7 and the pixel interval P2 of the special light imaging sensor 9 may be designed so satisfy the following conditional expression (2).

$$1 \leq \frac{P 2}{P 1} < 5 \qquad ( 2 )$$

The ratio (P2/P1) falling below 1 would cause a decrease in the pixel size W2 of the special light imaging sensor 9, a decrease in the quantity of light for capturing the weak special light FL into the pixel 9p, leading to an increase in noise in the captured image. The ratio (P2/P1) being 5 or more would cause an increase in the pixel size W2 of the special light imaging sensor 9, leading to enlargement of the imaging device 1. Decreasing the number of pixels 9p in order to prevent these situations would degrade the resolution.

The ratio (P2/P1) being greater than 1 will increase the pixel size W2 of the special light imaging sensor 9 and thus increase the quantity of incident light, making it possible to achieve reception of the weak special light FL with high sensitivity and imaging with high clarity. When the ratio (P2/P1) is 1, for example, by using a color imaging sensor for the visible light imaging sensor 7 and using a monochrome imaging sensor for the special light imaging sensor 9, it is possible to receive the special light FL with high sensitivity and image the special light FL with high clarity while maintaining the equivalent resolution.

Incidentally, the lower limit value of the ratio (P2/P1) may be greater than the value illustrated in the conditional expression (2). Examples of such lower limit values include 1.1, 1.2, 1.3, 1.4, 1.5, 1.6, 1.7, 1.8, 1.9, 2.0, 2.1, 2.2, 2.3, 2.4, and 2.5. The upper limit value may be smaller than the value indicated by the conditional expression (2). Examples of such upper limit values include 4.8, 4.6, 4.4, 4.2, 4.0, 3.8, 3.6, 3.4, 3.2, 3.0, 2.8, 2.6, 2.4, 2.2, 2.0, 1.8, and 1.6.

### <Design Condition 1-3>

In one embodiment, the ratio (D2/D1) of the effective pixel area diagonal length D2 of the special light imaging sensor 9 to the effective pixel area diagonal length D1 of the visible light imaging sensor 7 may be greater than 0.7 and less than 1.4. That is, the effective pixel area diagonal length D1 of the visible light imaging sensor 7 and the effective pixel area diagonal length D2 of the special light imaging sensor 9 may be designed to satisfy the following conditional expression (3).

$$0.7 \leq \frac{D 2}{D 1} < 1.4 \qquad ( 3 )$$

The ratio (D2/D1) falling to 0.7 or less would significantly decrease the angle of view of the special light imaging sensor 9 as compared with the angle of view of the visible light imaging sensor 7, restricting the field of view in which the special light FL can be observed. The ratio (D2/D1) of 1.4 or more would significantly increase the angle of view of the special light imaging sensor 9 as compared with the angle of view of the visible light imaging sensor 7, causing occurrence of pixels 9p that are not used in the effective pixel area 9a2 of the special light imaging sensor 9.

When the ratio (D2/D1) is smaller than 1, the special light imaging sensor 9 is relatively small with respect to the prism 6, and the optical path can have a sufficient margin. This makes it possible to reduce beams of unwanted light generated by the reflection of the members constituting the prism 6 and incident on the special light imaging sensor 9, leading to achievement of imaging of the weak special light FL with high clarity. For example, it is possible to reduce an unwanted light component such as flare occurring with an increased sensitivity of the special light imaging sensor 9, and possible to obtain a clear fluorescence image. When the ratio (D2/D1) is greater than 1, the pixel size W2 can be increased while keeping the number of pixels 9p relatively large, making it possible to image the weak special light FL with high sensitivity and high resolution. When the ratio (D2/D1) is 1, it is possible to obtain high sensitivity by increasing the pixel size W2 by increasing the pixel interval P2 of the special light imaging sensor 9, and further, it is possible to use the accompanying components as commonly usable components, facilitating the assemblability of the imaging device 1.

Incidentally, the lower limit value of the ratio (D2/D1) may be greater than the value illustrated in the conditional expression (2). Examples of such lower limit values include 0.8, 0.9, and 1.0. The upper limit value may be smaller than the value indicated by the conditional expression (3). Examples of such upper limit values include 1.3, 1.2, and 1.1.

### <Design Condition 1-4>I

The optical path lengths of the visible light VL and the special light FL from the incidence on the prism 6 to image forming may be different from each other depending on the conditions such as characteristics of the optical diagnosis assembly 3 and the imaging lens 4, and the wavelength of each light. In that case, the visible light imaging sensor 7 may be positioned at the image forming position of the visible light VL and the special light imaging sensor 9 may be positioned at the image forming position of the special light FL so as to suppress the influence of the difference between the optical path lengths (optical path length difference), in other words, to absorb the deviation amount of the optical path length difference. With this configuration, the special light imaging sensor 9 can achieve focusing only by focusing the visible light imaging sensor 7, facilitating imaging of the visible light VL with high resolution and imaging of the special light FL with high clarity. Some examples of specific positioning methods will be described.

### <Positioning Method 1-1>

In one embodiment, the band pass filter 8 may have an optical axis direction length designed in accordance with the above-described optical path length difference so as to position the special light imaging sensor 9 at the image forming position of the special light FL. When the incoming surface 8a of the band pass filter 8 is perpendicular to the optical axis, the length of the band pass filter 8 in the optical axis direction corresponds to the thickness of the band pass filter 8. By adjusting the thickness of the band pass filter 8, the special light imaging sensor 9 can be brought close to the prism 6 or away from the prism 6.

### <Positioning Method 1-2>

In one embodiment, the prism 6 may have an optical axis direction length designed in accordance with the above-described optical path length difference so as to position the visible light imaging sensor 7 at the image forming position of the visible light VL and position the special light imaging sensor 9 at the image forming position of the special light FL. For example, by adjusting the optical axis direction length of the block 61, the visible light imaging sensor 7 can be brought close to the prism 6 or away from the prism 6. By adjusting the optical axis direction length of the block 62, the special light imaging sensor 9 can be brought close to the prism 6 or away from the prism 6.

### <Positioning Method 1-3>

In one embodiment, the imaging device 1 may include a void located between the prism 6 and at least one of the visible light imaging sensor 7 and the special light imaging sensor 9. The void may have an optical axis direction length designed in accordance with the above-described optical path length difference so as to position the visible light imaging sensor 7 at the image forming position of the visible light VL and position the special light imaging sensor 9 at the image forming position of the special light FL. It is also allowable to use a holding member for providing such a void. This will be described with reference to FIG. 10.

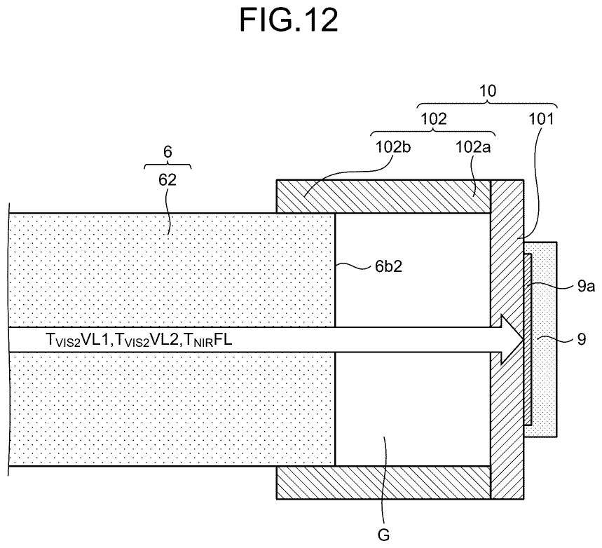

FIG. 12 is a diagram illustrating an example of the holding member. In this example, a holding member 10 provides a void G located between the block 62 of the prism 6 and the special light imaging sensor 9. Note that illustration of the band pass filter 8 (FIG. 1) is omitted. The void G can also be regarded as an air gap between the block 62 and the special light imaging sensor 9. The holding member 10 holds the special light imaging sensor 9 so as to provide the void G. Specifically, in this example, the holding member 10 includes a substrate 101 and a holding rod 102.

The substrate 101 is bonded to the light receiving surface 9a of the special light imaging sensor 9 to support the special light imaging sensor 9. An example of the material of the substrate 101 is glass. The special light FL transmitted through the substrate 101 is incident on the light receiving surface 9a of the special light imaging sensor 9. The holding rod 102 includes one end 102a connected to the edge portion of the substrate 101 and the other end 102b that holds the side surface of the block 62 of the prism 6. The length of the holding rod 102 gives the optical axis direction length of the void G. By adjusting the length of the holding rod 102, the special light imaging sensor 9 can be brought close to the prism 6 or away from the prism 6.

Note that the imaging device 1 may include the holding member 10 that holds the visible light imaging sensor 7 instead of or in addition to the special light imaging sensor 9 that holds the holding member 10.

### 2. Second Embodiment

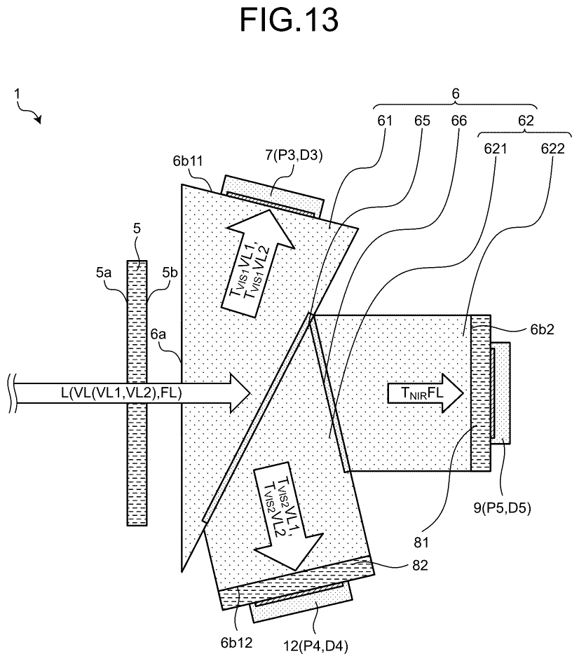

FIG. 13 is a diagram illustrating an example of a schematic configuration of an imaging device according to a second embodiment. The observation target S, the light source 2, the optical diagnosis assembly 3, the imaging lens 4, and the processing unit 11 in FIG. 1 described above are not illustrated in FIG. 13. Differences from the first embodiment will be mainly described. The prism 6 further includes a dichroic film 66. The block 62 of the prism 6 includes a block 621 and a block 622. The block 621 is located between the block 61 and the block 621. The imaging device 1 includes a band pass filter 81 and a band pass filter 82 instead of the band pass filter 8 (FIG. 1), and further includes a visible special light imaging sensor 12.

The prism 6 further separates and outputs the visible light VL (normal visible light VL1 and visible special light VL2) and the special light FL transmitted through the dichroic film 65. The surface from which the visible light VL transmitted through the dichroic film 65 is emitted is denoted as an outgoing surface 6b12 in the drawing. The surface from which the special light FL is emitted is an outgoing surface 6b2. An outgoing surface 6b11 is a part of the surface of the block 61. The outgoing surface 6b12 is a part of the surface of the block 621. The surface from which the special light FL is emitted is the outgoing surface 6b2. The outgoing surface 6b2 is a part of the surface of the block 622.

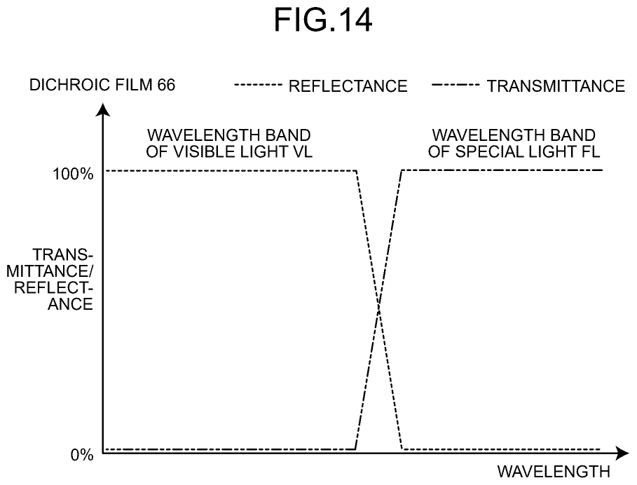

The dichroic film 66 is provided between the block 621 and the block 622 so that the visible light VL (normal visible light VL1 and visible special light VL2) and the special light FL transmitted through the dichroic film 65 are to be incident. In this example, the dichroic film 66 is deposited on the block 621. The dichroic film 66 reflects the normal visible light VL1 and the visible special light VL2 and transmits the special light FL. This will be described with reference to FIG. 14.

FIG. 14 is a diagram illustrating an example of characteristics of the dichroic film 66. The reflectance is high at least in the wavelength band including the visible light VL, while the transmittance is high at least in the wavelength band including the special light FL. The dichroic film 65 is designed to impart spectral transmittance characteristics of the prism 6 described below.

Returning to FIG. 13, the block 621 of the block 62 of the prism 6 guides the visible light VL (normal visible light VL1 and visible special light VL2) to the visible special light imaging sensor 12. The block 621 emits the visible light VL and the visible special light VL2 reflected by the dichroic film 66 from the outgoing surface 6b12. The block 622 guides the special light FL to the special light imaging sensor 9. The block 622 emits the special light FL transmitted through the dichroic film 66, from the outgoing surface 6b2.

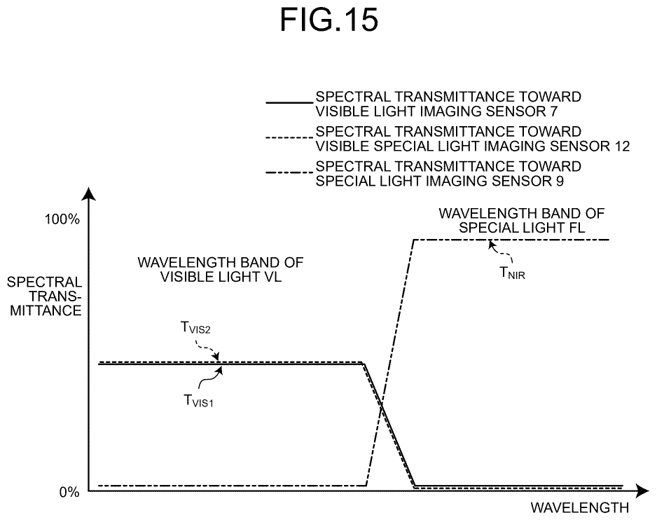

The spectral transmittance T_{VIS2} in the second embodiment is the spectral transmittance of the visible light VL toward the visible special light imaging sensor 12 in the prism 6. This will be described with reference to FIG. 15.

FIG. 15 is a diagram illustrating an example of spectral transmittance of a prism. The spectral transmittance toward the visible special light imaging sensor 12 in the wavelength band of the visible light VL is the spectral transmittance T_{VIS2}. The spectral transmittance T_{VIS1} and the spectral transmittance T_{NIR} are as described above. The spectral transmittance T_{VIS1} and the spectral transmittance T_{VIS2} are values apart from 0% and 100%.

Returning to FIG. 13, the normal visible light VL1 emitted from the outgoing surface 6b12 of the prism 6 has a light quantity (T_{VIS2}VL1) obtained by multiplying the light quantity of the normal visible light VL1 incident on the incoming surface 6a by the spectral transmittance T_{VIS2}. Similarly, the visible special light VL2 emitted has a light quantity (T_{VIS2}VL2) obtained by multiplying the light quantity of the visible special light VL2 incident on the incoming surface 6a by the spectral transmittance T_{VIS2}.

The band pass filter 81 and the band pass filter 82 will be described in order. The special light FL from the outgoing surface 6b2 of the prism 6 is incident on the band pass filter 81.

The band pass filter 81 is a first band pass filter provided between the block 622 of the block 62 of the prism 6 and the special light imaging sensor 9. The band pass filter 81 transmits the special light FL and reflects or attenuates other light. The incoming surface of the band pass filter 81 may be a surface perpendicular to the optical axis. The band pass filter 81 is provided for the prism 6 so that the incoming surface faces the outgoing surface 6b2 of the prism 6. In the example illustrated in FIG. 13, the band pass filter 81 is provided (bonded, for example) on the outgoing surface of the block 622 of the block 62 of the prism 6. The special light FL from the outgoing surface of the band pass filter 81 forms an image, which is captured by the special light imaging sensor 9.

The normal visible light VL1 and the visible special light VL2 from the outgoing surface 6b12 of the prism 6 are incident on the band pass filter 82.

The band pass filter 82 is a second band pass filter provided between the block 621 of the block 62 of the prism 6 and the visible special light imaging sensor 12. The band pass filter 82 transmits the visible special light VL2 and reflects or attenuates the normal visible light VL1 belonging to a wavelength band different from the wavelength band of the visible special light VL2. The band pass filter 82 may be designed to reflect or attenuate the special light FL. The incoming surface of the band pass filter 82 may be a surface perpendicular to the optical axis. The band pass filter 82 is provided for the prism 6 so that the incoming surface faces the outgoing surface 6b12 of the prism 6. In the example illustrated in FIG. 13, the band pass filter 82 is provided (bonded, for example) on the outgoing surface of the block 622 of the block 62 of the prism 6.

The visible special light VL2 from the outgoing surface of the band pass filter 82 forms an image, which is captured by the visible special light imaging sensor 12. The visible special light imaging sensor 12 is directly or indirectly fixed to the prism 6 so as to be located at the image forming position of the visible special light VL2.

The visible special light imaging sensor 12 images the visible special light VL2 included in the visible light VL from the block 621 of the block 62 of the prism 6, that is, the visible special light VL2 from the outgoing surface of the band pass filter 82. The visible special light imaging sensor 12 is, for example, a Complementary Metal Oxide Semiconductor (CMOS) image sensor designed to image the visible special light VL2.

In the second embodiment, the pixel interval and the effective pixel area diagonal length of the visible light imaging sensor 7 are denoted as a pixel interval P3 and an effective pixel area diagonal length D3, respectively. The pixel interval and the effective pixel area diagonal length of the visible special light imaging sensor 12 are denoted as a pixel interval P4 and an effective pixel area diagonal length D4, respectively. The pixel interval and the effective pixel area diagonal length of the special light imaging sensor 9 are denoted as a pixel interval P5 and an effective pixel area diagonal length D5, respectively.

The imaging device 1 according to the second embodiment is designed to be able to image the visible special light VL2 with high clarity even when observing both the normal visible light VL1 and the visible special light VL2. In addition, the imaging device 1 may be designed so to achieve imaging of the normal visible light VL1 with high resolution and achieve imaging of the special light FL with high clarity as well. Several design conditions will be described. Among the design conditions described below, non-exclusive design conditions may be flexibly combined.

### <Design Condition 2-1>

In one embodiment, the ratio (T_{VIS2}/T_{VIS1}) of the spectral transmittance T_{VIS2} of the visible light VL toward the visible special light imaging sensor 12 to the spectral transmittance T_{VIS1} of the visible light VL toward the visible light imaging sensor 7 in the prism 6 may be 0.2 or more and less than 5. The spectral transmittance T_{NIR} of the special light FL for the special light imaging sensor 9 in the prism 6 may be greater than 90%. That is, the dichroic film 65 and the dichroic film 66 of the prism 6 may be designed so as to satisfy the following conditional expression (4). Since the technical significance is similar to the conditional expression (1) described in the first embodiment, the description thereof is omitted. The similar applies to the point that the lower limit value may be greater than the value indicated by the conditional expression and the point that the upper limit value may be smaller than the value indicated by the conditional expression. Further examples of the lower limit values are 2.25 and 2.5. Further examples of the upper limit value include 4.75, 4.5, 4.25, and 4.0.

$$0.2 \leq \frac{{T}_{V I S 2}}{{T}_{V I S 1}} \leq 5 and {T}_{NIR} > 90 \% \qquad ( 4 )$$

### <Design Condition 2-2>

In one embodiment, both the ratio (P4/P3) of the pixel interval P4 of the visible special light imaging sensor 12 to the pixel interval P3 of the visible light imaging sensor 7 and the ratio (P5/P3) of the pixel interval P5 of the special light imaging sensor 9 to the pixel interval P3 of the visible light imaging sensor 7 may be 1 or more and less than 5. That is, the pixel interval P3 of the visible light imaging sensor 7, the pixel interval P4 of the visible special light imaging sensor 12, and the pixel interval P5 of the special light imaging sensor 9 may be designed so as to satisfy the following conditional expression (5). Since the technical significance is similar to the conditional expression (2) described in the first embodiment, the description thereof is omitted. The similar applies to the point that the lower limit value may be greater than the value indicated by the conditional expression and the point that the upper limit value may be smaller than the value indicated by the conditional expression.

$$1 \leq \frac{P 4}{P 3} < 5 and 1 \leq \frac{P 5}{P 3} < 5 \qquad ( 5 )$$

### <Design Condition 2-3>

In one embodiment, both the ratio (D4/D3) of the effective pixel area diagonal length D4 of the visible special light imaging sensor 12 to the effective pixel area diagonal length D3 of the visible light imaging sensor 7 and the ratio (D5/D3) of the effective pixel area diagonal length D5 of the special light imaging sensor 9 to the effective pixel area diagonal length D3 of the visible light imaging sensor 7 may be greater than 0.7 and less than 1.4. That is, the effective pixel area diagonal length D3 of the visible light imaging sensor 7, the effective pixel area diagonal length D4 of the visible special light imaging sensor 12, and the effective pixel area diagonal length D5 of the special light imaging sensor 9 may be designed to satisfy the following conditional expression (6). Since the technical significance is similar to the conditional expression (3) described in the first embodiment, the description thereof is omitted. The similar applies to the point that the lower limit value may be greater than the value indicated by the conditional expression and the point that the upper limit value may be smaller than the value indicated by the conditional expression.

$$0.7 \leq \frac{D 4}{D 3} < 1.4 and 0.7 < \frac{D 5}{D 3} < 1.4$$

### <Design Condition 2-4>

The optical path lengths of the normal visible light VL1, the visible special light VL2, and the special light FL from the incident on the prism 6 to image forming may be different from each other depending on the conditions such as the characteristics of the optical diagnosis assembly 3 and the imaging lens 4, and the wavelength of each beams of light. In that case, the visible light imaging sensor 7 may be positioned at the image forming position of the visible light VL, the special light imaging sensor 9 may be positioned at the image forming position of the special light FL, and the visible special light imaging sensor 12 may be positioned at the image forming position of the visible special light VL2 so as to suppress the influence of these optical path length differences. With this configuration, the special light imaging sensor 9 and the visible special light imaging sensor 12 can achieve focusing only by focusing the visible light imaging sensor 7, facilitating imaging of the normal visible light VL1 with high resolution and imaging of the special light FL and the visible special light VL2 with high clarity. Some examples of specific positioning methods will be described.

### <Positioning Method 2-1>

In one embodiment, the band pass filter 81 may have an optical axis direction length designed in accordance with an optical path length difference between the normal visible light VL1 and the special light FL, for example, so as to position the special light imaging sensor 9 at the image forming position of the special light FL. Similarly, the band pass filter 82 may have an optical axis direction length designed in accordance with the optical path length difference between the normal visible light VL1 and the visible special light VL2, for example, so as to position the visible special light imaging sensor 12 at the image forming position of the visible special light VL2.

### <Positioning Method 2-2>

In one embodiment, the prism 6 may have an optical axis direction length designed in accordance with the above-described optical path length difference so as to position the visible light imaging sensor 7 at the image forming position of the visible light VL (for example, the image forming position of the normal visible light VL1) and so as to position the special light imaging sensor 9 at the coupling position of the special light FL, and position the visible special light imaging sensor 12 at the image forming position of the visible special light VL2. For example, by adjusting the optical axis direction length of the block 61, the visible light imaging sensor 7 can be brought close to the prism 6 or away from the prism 6. By adjusting the optical axis direction length of the block 622, the special light imaging sensor 9 can be brought close to the prism 6 or away from the prism 6. By adjusting the optical axis direction length of the block 621, the visible special light imaging sensor 12 can be brought close to the prism 6 or away from the prism 6.

### <Positioning Method 2-3>

In one embodiment, the imaging device 1 may include a void located between the prism 6 and at least one of the visible light imaging sensor 7, the special light imaging sensor 9, and the visible special light imaging sensor 12. The void may have an optical axis direction length designed in accordance with the above-described optical path length difference so as to position the visible light imaging sensor 7 at the image forming position of the visible light VL, position the special light imaging sensor 9 at the image forming position of the special light FL, and position the visible special light imaging sensor 12 at the image forming position of the visible special light VL2. It is also allowable to use a holding member for providing such a void. The void and the holding member may be similar to those described in the first embodiment with reference to FIG. 12.

### 3. Modifications

The disclosed technology is not limited to the above embodiments. Some modifications will be described.

The position of the imaging sensor may be appropriately interchanged. For example, in the first embodiment, the positions of the visible light imaging sensor 7 and the special light imaging sensor 9 may be interchanged. In accordance with the change, conditions such as the characteristics of the dichroic film 65 of the prism 6 and the arrangement of the band pass filter 8 may also be changed. In the second embodiment, the positions of the visible light imaging sensor 7, the special light imaging sensor 9, and the visible special light imaging sensor 12 may be interchanged. In accordance with the change, conditions such as the characteristics of the dichroic film 65 and the dichroic film 66 of the prism 6, and the arrangement of the band pass filter 81 and the band pass filter 82, may also be changed.

As described above, there may be a void (for example, an air gap) between the prism 6 and the visible light imaging sensor 7, between the band pass filter 8 of the prism 6, or between the band pass filter 8 and the visible light imaging sensor 7. In addition, the optical element 5 may be bonded to the block 61 of the prism 6. There may be an air gap between the blocks included in the prism 6.

### 4. Application Example

The technology according to the present disclosure can be applied to a medical imaging system. The medical imaging system is a medical system using an imaging technology, and includes a medical observation system such as an endoscope system or a microscope system, for example.

### <Endoscope System>

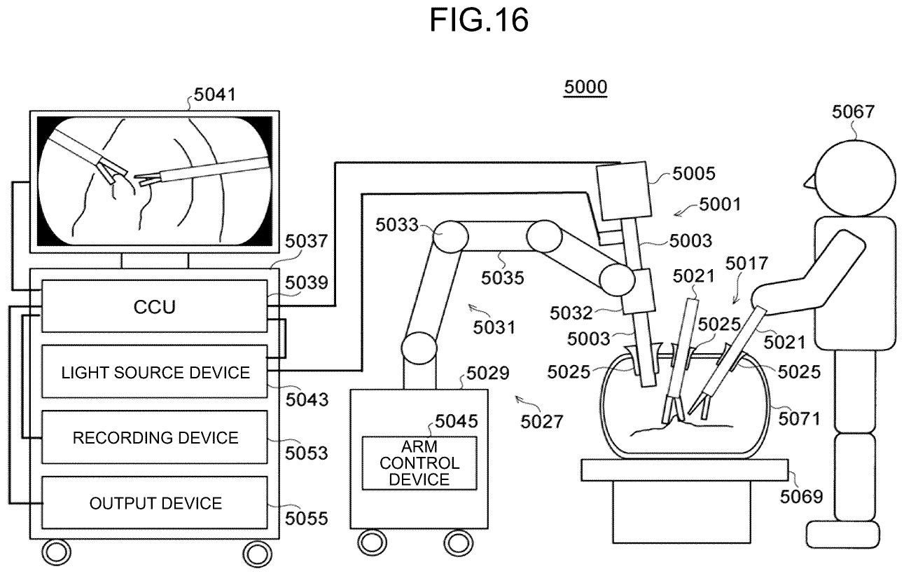

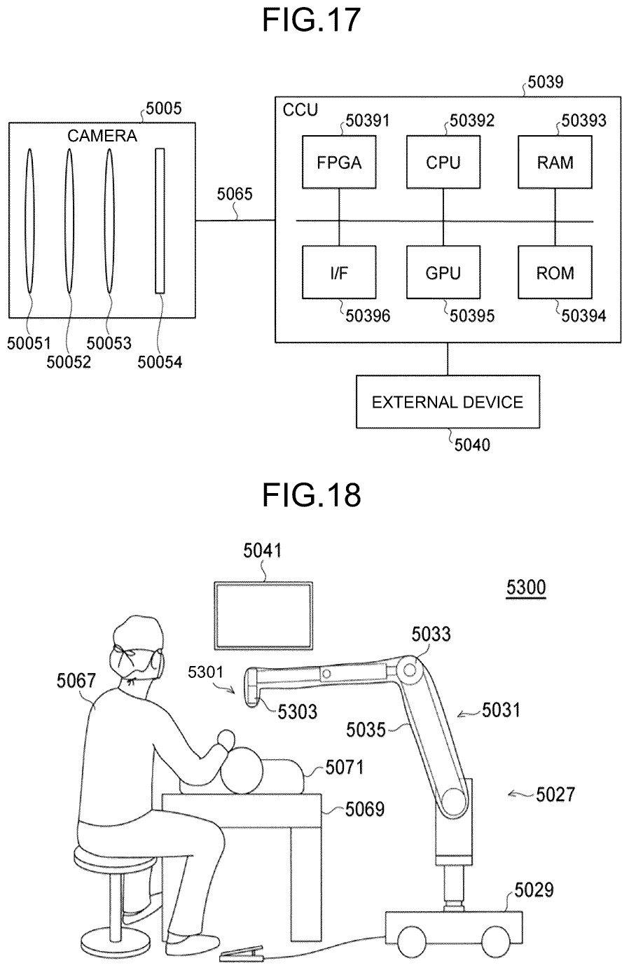

An example of the endoscope system will be described using FIGS. 16 and 17. FIG. 16 is a diagram illustrating an example of a schematic configuration of an endoscope system 5000 to which the technology according to the present disclosure is applicable. FIG. 17 is a diagram illustrating an example of a configuration of an endoscope 5001 and a camera control unit (CCU) 5039. FIG. 17 illustrates a situation where an operator (for example, a doctor) 5067 who is a participant of an operation performs the operation on a patient 5071 on a patient bed 5069 using the endoscope system 5000. As illustrated in FIG. 17, the endoscope system 5000 includes the endoscope 5001 that is a medical imaging device, the CCU 5039, a light source device 5043, a recording device 5053, an output device 5055, and a support device 5027 for supporting the endoscope 5001.

In endoscopic surgery, insertion assisting tools called trocars 5025 are punctured into the patient 5071. Then, a scope 5003 connected to the endoscope 5001 and surgical tools 5021 are inserted into a body of the patient 5071 through the trocars 5025. The surgical tools 5021 include: an energy device such as an electric scalpel; and forceps, for example.

A surgical image that is a medical image in which the inside of the body of the patient 5071 is captured by the endoscope 5001 is displayed on a display device 5041. The operator 5067 performs a procedure on a surgical target using the surgical tools 5021 while viewing the surgical image displayed on the display device 5041. The medical image is not limited to the surgical image, and may be a diagnostic image captured during diagnosis.

### <Endoscope>

The endoscope 5001 is an imaging section for capturing the inside of the body of the patient 5071, and is, for example, as illustrated in FIG. 18, a camera 5005 including a condensing optical system 50051 for condensing incident light, a zooming optical system 50052 capable of optical zooming by changing a focal length of the imaging section, a focusing optical system 50053 capable of focus adjustment by changing the focal length of the imaging section, and a light receiving sensor 50054. The endoscope 5001 condenses the light through the connected scope 5003 on the light receiving sensor 50054 to generate a pixel signal, and outputs the pixel signal through a transmission system to the CCU 5039. The scope 5003 is an insertion part that includes an objective lens at a distal end and guides the light from the connected light source device 5043 into the body of the patient 5071. The scope 5003 is, for example, a rigid scope for a rigid endoscope and a flexible scope for a flexible endoscope. The scope 5003 may be a direct viewing scope or an oblique viewing scope. The pixel signal only needs to be a signal based on a signal output from a pixel, and is, for example, a raw signal or an image signal. The transmission system connecting the endoscope 5001 to the CCU 5039 may include a memory, and the memory may store parameters related to the endoscope 5001 and the CCU 5039. The memory may be disposed at a connection portion of the transmission system or on a cable. For example, the memory of the transmission system may store the parameters before shipment of the endoscope 5001 or the parameters changed when current is applied, and an operation of the endoscope may be changed based on the parameters read from the memory. A set of the camera and the transmission system may be referred to as an endoscope. The light receiving sensor 50054 is a sensor for converting the received light into the pixel signal, and is, for example, a complementary metal-oxide-semiconductor (CMOS) imaging sensor. The light receiving sensor 50054 is preferably an imaging sensor having a Bayer array capable of color imaging. The light receiving sensor 50054 is also preferably an imaging sensor having a number of pixels corresponding to a resolution of, for example, 4K (3840 horizontal pixels×2160 vertical pixels), 8K (7680 horizontal pixels×4320 vertical pixels), or square 4K (3840 or more horizontal pixels×3840 or more vertical pixels). The light receiving sensor 50054 may be one sensor chip, or a plurality of sensor chips. For example, a prism may be provided to separate the incident light into predetermined wavelength bands, and the wavelength bands may be imaged by different light receiving sensors. A plurality of light receiving sensors may be provided for stereoscopic viewing. The light receiving sensor 50054 may be a sensor having a chip structure including an arithmetic processing circuit for image processing, or may be a sensor for time of flight (ToF). The transmission system is, for example, an optical fiber cable system or a wireless transmission system. The wireless transmission only needs to be capable of transmitting the pixel signal generated by the endoscope 5001, and, for example, the endoscope 5001 may be wirelessly connected to the CCU 5039, or the endoscope 5001 may be connected to the CCU 5039 via a base station in an operating room. At this time, the endoscope 5001 may transmit not only the pixel signal, but also simultaneously information (for example, a processing priority of the pixel signal and/or a synchronization signal) related to the pixel signal. In the endoscope, the scope may be integrated with the camera, and the light receiving sensor may be provided at the distal end of the scope.

### <CCU (Camera Control Unit)>

The CCU 5039 is a control device for controlling the endoscope 5001 and the light source device 5043 connected to the CCU 5039 in an integrated manner, and is, for example, as illustrated in FIG. 18, an image processing device including a field-programmable gate array (FPGA) 50391, a central processing unit (CPU) 50392, a random access memory (RAN) 50393, a read-only memory (ROM) 50394, a graphics processing unit (GPU) 50395, and an interface (I/F) 50396. The CCU 5039 may control the display device 5041, the recording device 5053, and the output device 5055 connected to the CCU 5039 in an integrated manner. The CCU 5039 controls, for example, irradiation timing, irradiation intensity, and a type of an irradiation light source of the light source device 5043. The CCU 5039 also performs image processing, such as development processing (for example, demosaic processing) and correction processing, on the pixel signal output from the endoscope 5001, and outputs the processed image signal (for example, an image) to an external device such as the display device 5041. The CCU 5039 also transmits a control signal to the endoscope 5001 to control driving of the endoscope 5001. The control signal is information on an imaging condition such as a magnification or the focal length of the imaging section. The CCU 5039 may have a function to down-convert the image, and may be configured to be capable of simultaneously outputting a higher-resolution (for example, 4K) image to the display device 5041 and a lower-resolution (for example, high-definition (HD)) image to the recording device 5053.

The CCU 5039 may be connected to external equipment (such as a recording device, a display device, an output device, and a support device) via an IP converter for converting the signal into a predetermined communication protocol (such as the Internet Protocol (IP)). The connection between the IP converter and the external equipment may be established using a wired network, or a part or the whole of the network may be established using a wireless network. For example, the IP converter on the CCU 5039 side may have a wireless communication function, and may transmit the received image to an IP switcher or an output side IP converter via a wireless communication network, such as the fifth-generation mobile communication system (5G) or the sixth-generation mobile communication system (6G).

### <Light Source Device>

The light source device 5043 is a device capable of emitting the light having predetermined wavelength bands, and includes, for example, a plurality of light sources and a light source optical system for guiding the light of the light sources. The light sources are, for example, xenon lamps, light-emitting diode (LED) light sources, or laser diode (LD) light sources. The light source device 5043 includes, for example, the LED light sources corresponding to three respective primary colors of red (R), green (G), and blue (B), and controls output intensity and output timing of each of the light sources to emit white light. The light source device 5043 may include a light source capable of emitting special light used for special light observation, in addition to the light sources for emitting normal light for normal light observation. The special light is light having a predetermined wavelength band different from that of the normal light being light for the normal light observation, and is, for example, near-infrared light (light having a wavelength of 760 nm or longer), infrared light, blue light, or ultraviolet light. The normal light is, for example, the white light or green light. In narrow band imaging that is a kind of special light observation, blue light and green light are alternately emitted, and thus the narrow band imaging can image a predetermined tissue such as a blood vessel in a mucosal surface at high contrast using wavelength dependence of light absorption in the tissue of the body. In fluorescence observation that is a kind of special light observation, excitation light is emitted for exciting an agent injected into the tissue of the body, and fluorescence emitted by the tissue of the body or the agent as a label is received to obtain a fluorescent image, and thus the fluorescence observation can facilitate the operator to view, for example, the tissue of the body that is difficult to be viewed by the operator with the normal light. For example, in fluorescence observation using the infrared light, the infrared light having an excitation wavelength band is emitted to an agent, such as indocyanine green (ICG), injected into the tissue of the body, and the fluorescence light from the agent is received, whereby the fluorescence observation can facilitate viewing of a structure and an affected part of the tissue of the body. In the fluorescence observation, an agent (such as 5-aminolevulinic acid (5-ALA)) may be used that emits fluorescence in a red wavelength band by being excited by the special light in a blue wavelength band. The type of the irradiation light of the light source device 5043 is set by control of the CCU 5039. The CCU 5039 may have a mode of controlling the light source device 5043 and the endoscope 5001 to alternately perform the normal light observation and the special light observation. At this time, information based on a pixel signal obtained by the special light observation is preferably superimposed on a pixel signal obtained by the normal light observation. The special light observation may be an infrared light observation to observe a site inside the surface of an organ and a multi-spectrum observation utilizing hyperspectral spectroscopy. A photodynamic therapy may be incorporated.

### <Recording Device>

The recording device 5053 is a device for recording the pixel signal (for example, an image) acquired from the CCU 5039, and is, for example, a recorder. The recording device 5053 records an image acquired from the CCU 5039 in a hard disk drive (HDD), a Super Density Disc (SDD), and/or an optical disc. The recording device 5053 may be connected to a network in a hospital to be accessible from equipment outside the operating room. The recording device 5053 may have a down-convert function or an up-convert function.

### <Display Device>

The display device 5041 is a device capable of displaying the image, and is, for example, a display monitor. The display device 5041 displays a display image based on the pixel signal acquired from the CCU 5039. The display device 5041 may include a camera and a microphone to function as an input device that allows instruction input through gaze recognition, voice recognition, and gesture.

### <Output Device>

The output device 5055 is a device for outputting the information acquired from the CCU 5039, and is, for example, a printer. The output device 5055 prints, for example, a print image based on the pixel signal acquired from the CCU 5039 on a sheet of paper.

### <Support Device>

The support device 5027 is an articulated arm including a base 5029 including an arm control device 5045, an arm 5031 extending from the base 5029, and a holding part 5032 mounted at a distal end of the arm 5031. The arm control device 5045 includes a processor such as a CPU, and operates according to a predetermined computer program to control driving of the arm 5031. The support device 5027 uses the arm control device 5045 to control parameters including, for example, lengths of links 5035 constituting the arm 5031 and rotation angles and torque of joints 5033 so as to control, for example, the position and attitude of the endoscope 5001 held by the holding part 5032. This control can change the position or attitude of the endoscope 5001 to a desired position or attitude, makes it possible to insert the scope 5003 into the patient 5071, and can change the observed area in the body. The support device 5027 functions as an endoscope support arm for supporting the endoscope 5001 during the operation. Thus, the support device 5027 can play a role of a scopist who is an assistant holding the endoscope 5001. The support device 5027 may be a device for holding a microscope device 5301 to be described later, and can be called a medical support arm. The support device 5027 may be controlled using an autonomous control method by the arm control device 5045, or may be controlled using a control method in which the arm control device 5045 performs the control based on input of a user. The control method may be, for example, a master-slave method in which the support device 5027 serving as a slave device (replica device) that is a patient cart is controlled based on a movement of a master device (primary device) that is an operator console at a hand of the user. The support device 5027 may be remotely controllable from outside the operating room.

The example of the endoscope system 5000 to which the technology according to the present disclosure is applicable has been described above. For example, the technology according to the present disclosure may be applied to a microscope system.

### <Microscope System>

FIG. 18 is a diagram illustrating an example of a schematic configuration of a microscopic surgery system to which the technology according to the present disclosure is applicable. In the following description, the same components as those of the endoscope system 5000 will be denoted by the same reference numerals, and the description thereof will not be repeated.

FIG. 18 schematically illustrates a situation where the operator 5067 performs an operation on the patient 5071 on the patient bed 5069 using a microscopic surgery system 5300. For the sake of simplicity, FIG. 18 does not illustrate a cart 5037 among the components of the microscopic surgery system 5300, and illustrates the microscope device 5301 instead of the endoscope 5001 in a simplified manner. The microscope device 5301 may refer to a microscope 5303 provided at the distal end of the links 5035, or may refer to the overall configuration including the microscope 5303 and the support device 5027.

As illustrated in FIG. 18, during the operation, the microscopic surgery system 5300 is used to display an image of a surgical site captured by the microscope device 5301 in a magnified manner on the display device 5041 installed in the operating room. The display device 5041 is installed in a position facing the operator 5067, and the operator 5067 performs various procedures, such as excision of an affected part, on the surgical site while observing the state of the surgical site using the image displayed on the display device 5041. The microscopic surgery system is used in, for example, ophthalmic operation and neurosurgical operation.

The respective examples of the endoscope system 5000 and the microscopic surgery system 5300 to which the technology according to the present disclosure is applicable have been described above. Systems to which the technology according to the present disclosure is applicable are not limited to such examples. For example, the support device 5027 can support, at the distal end thereof, another observation device or another surgical tool instead of the endoscope 5001 or the microscope 5303. Examples of the other applicable observation device include forceps, tweezers, a pneumoperitoneum tube for pneumoperitoneum, and an energy treatment tool for incising a tissue or sealing a blood vessel by cauterization. By using the support device to support the observation device or the surgical tool described above, the position thereof can be more stably fixed and the load of the medical staff can be lower than in a case where the medical staff manually supports the observation device or the surgical tool. The technology according to the present disclosure may be applied to a support device for supporting such a component other than the microscope.

The imaging device 1 according to each embodiment described above can be suitably applied to the endoscope 5001 and the microscope device 5301 among the above-described configurations. Specifically, the observation target S is a surgical site. The light source 2 can constitute the light source device 5043. The optical diagnosis assembly 3 can constitute a part of the endoscope 5001 or the microscope device 5301. Portions including the imaging lens 4, the optical element 5, the prism 6, the visible light imaging sensor 7, the band pass filter 8, and the fluorescence imaging sensor 9 can function as the imaging section. By using the imaging device 1, it is possible to image the visible special light with high clarity.

### 5. Exemplary Effects

The technology described above is specified as follows, for example. One of the disclosed technologies is the imaging device 1. As described with reference to diagrams such as FIGS. 1 to 3, 5 to 11, the imaging device 1 includes: the prism 6 that separates the visible light VL (including the normal visible light VL1 and the visible special light VL2, for example) and the special light FL included in the light L from the observation target S; the visible light imaging sensor 7 that images the visible light VL separated by the prism 6; and the special light imaging sensor 9 that images the special light FL separated by the prism 6. The ratio (T_{VIS2}/T_{VIS1}) of the spectral transmittance T_{VIS2} of the visible light VL toward the special light imaging sensor 9 to the spectral transmittance T_{VIS1} of the visible light VL toward the visible light imaging sensor 7 in the prism 6 is 0.25 or more and 4 or less. The spectral transmittance T_{NIR} of the special light FL toward the special light imaging sensor 9 in the prism 6 is greater than 90%. By designing the spectral transmittance T_{VIS1}, the spectral transmittance T_{VIS2}, and the spectral transmittance T_{NIR} of the prism 6 in this manner, when the visible light VL includes the normal visible light VL1 and the visible special light VL2, and both of the beams of light is to be observed, the visible special light VL2 can be imaged with high clarity.

As described with reference to diagrams such as FIG. 1, the imaging device 1 may include a depolarizer (optical element 5) provided between the observation target S and the prism 6. This makes it possible to suppress the fluctuation in the quantity of output light caused by the polarization dependence of the spectral transmittance of the prism 6, enabling imaging of the visible light VL and the special light FL with stable exposure.

As described with reference to diagrams such as FIGS. 1 and 8, the imaging device 1 may include the band pass filter 8 that is provided between the prism 6 and the special light imaging sensor 9 and passes the visible special light VL2 and the special light FL, and the special light imaging sensor 9 may also image the visible special light VL2. By imaging the visible special light VL2 not only by the visible light imaging sensor 7 but also by the special light imaging sensor 9, the visible special light VL2 can be received with high sensitivity and imaged with high clarity.

As described with reference to diagrams such as FIGS. 1, 5 to 7 and 9 to 11, the ratio (P2/P1) of the pixel interval P2 of the special light imaging sensor 9 to the pixel interval P1 of the visible light imaging sensor 7 may be 1 or more and less than 5 (1≤(P2/P1)<5). By designing the pixel interval P1 of the visible light imaging sensor 7 and the pixel interval P2 of the special light imaging sensor 9 in this manner, it is possible to image the visible light VL with high resolution and image the special light FL with high clarity.

As described with reference to diagrams such as FIGS. 1, 5, and 9, the ratio (D2/D1) of the effective pixel area diagonal length D2 of the special light imaging sensor 9 to the effective pixel area diagonal length D1 of the visible light imaging sensor 7 may be greater than 0.7 and less than 1.4. The special light FL can be received with high sensitivity and can be imaged with high clarity in this manner, for example.

As described with reference to diagrams such as FIG. 1, the light L after passing through the imaging lens 4 may be incident on the prism 6, the visible light VL and the special light FL may have individually different optical path lengths from the incidence of the light L on the prism 6 to image forming, the visible light imaging sensor 7 may be directly or indirectly fixed to the prism 6 so as to be located at the image forming position of the visible light VL, and the special light imaging sensor 9 may be directly or indirectly fixed to the prism 6 so as to be located at the image forming position of the special light FL. With this configuration, the special light imaging sensor 9 can achieve focusing only by focusing the visible light imaging sensor 7, facilitating imaging of the visible light VL with high resolution and clear imaging of the special light FL.

As described with reference to diagrams such as FIGS. 1 and 12, the imaging device 1 may include the band pass filter 8 that is provided between the prism 6 and the special light imaging sensor 9 and transmits the visible special light VL2 and the special light FL, and the band pass filter 8 may have an optical axis direction length designed in accordance with an optical path length difference between optical path lengths of the visible light VL and the special light Fl so as to position the special light imaging sensor 9 at the image forming position of the special light FL. The prism 6 may have an optical axis direction length designed in accordance with the optical path length difference between the optical path lengths of the visible light VL and the special light FL so as to position the visible light imaging sensor 7 at the image forming position of the visible light VL and position the special light imaging sensor 9 at the image forming position of the special light FL. The imaging device 1 may include the void G positioned between the prism 6 and at least one of the visible light imaging sensor 7 and the special light imaging sensor 9, and the void G may have an optical axis direction length designed in accordance with an optical path length difference between optical path lengths of the visible light VL and the special light FL so as to position the visible light imaging sensor 7 at the image forming position of the visible light VL and position the special light imaging sensor 9 at the image forming position of the special light FL. The visible light imaging sensor 7 and the special light imaging sensor 9 can be positioned in this manner, for example.

As described with reference to diagrams such as FIG. 1, the imaging device 1 may include: the light source 2 that irradiates the observation target S with the white light WL and the excitation light EL1 at different timings from each other; and the signal processing section 113 that processes the imaging signal from the visible light imaging sensor 7. The normal visible light VL1 may include light obtained by reflecting the white light WL from the light source 2 on the observation target S, the visible special light VL2 may include light emitted by the observation target S excited by the irradiation of the excitation light EL1 from the light source 2, and the signal processing section 113 may process the imaging signal when the observation target S is irradiated with the white light WL as the normal visible light image signal, and may process the imaging signal when the observation target S is irradiated with the excitation light EL1 as the visible special light image signal. The normal visible light VL1 incident on the visible light imaging sensor 7 and the visible special light VL2 incident on the special light imaging sensor 9 can be imaged in time division in this manner, for example.

The imaging device 1 described with reference to diagrams such as FIGS. 13 to 15 is also one of the disclosed technologies. The imaging device 1 includes: the prism 6 that separates the visible light (for example, including the normal visible light VL1 and the visible special light VL2) and the special light FL included in the light L from the observation target S; the visible light imaging sensor 7 that images the visible light VL separated by the prism 6; the special light imaging sensor 9 that images the special light FL separated by the prism 6; and the visible special light imaging sensor 12 that images the visible special light VL2 included in the visible light VL separated by the prism 6. The ratio of the spectral transmittance T_{VIS2} of the visible light toward the visible special light imaging sensor 12 to the spectral transmittance T_{VIS1} of the visible light VL toward the visible light imaging sensor 7 in the prism 6 is greater than 0.2 and less than 5, and the spectral transmittance of the special light FL toward the special light imaging sensor 9 in the prism 6 is greater than 90%. By designing the spectral transmittance T_{VIS1}, the spectral transmittance T_{VIS2}, and the spectral transmittance T_{NIR} of the prism 6 in this manner, it is also possible to image the visible special light VL2 with high clarity.

As described with reference to diagrams such as FIG. 13, the imaging device 1 may include the depolarizer (optical element 5) provided between the observation target S and the prism 6, or may include the band pass filter 81 provided between the prism 6 and the visible special light imaging sensor 12 so as to allow passage of the visible special light VL2. This enables imaging of the visible light VL and the special light FL with stable exposure, and imaging of the visible special light VL2 with high clarity.

As described with reference to FIG. 13, etc., both the ratio (P4/P3) of the pixel interval P4 of the visible special light imaging sensor 12 to the pixel interval P3 of the visible light imaging sensor 7 and the ratio (P5/P3) of the pixel interval P5 of the special light imaging sensor 9 to the pixel interval P3 of the visible light imaging sensor 7 may be 1 or more and less than 5. With this configuration, the visible light VL can be imaged at high resolution, and the special light FL can be imaged with high clarity.

As described with reference to diagrams such as FIG. 13, both the ratio (D4/D3) of the effective pixel area diagonal length D4 of the visible special light imaging sensor 12 to the effective pixel area diagonal length D3 of the visible light imaging sensor 7 and the ratio of the effective pixel area diagonal length D5 of the special light imaging sensor 9 to the effective pixel area diagonal length D3 of the visible light imaging sensor 7 may be greater than 0.7 and less than 1.4. With this configuration, the special light FL can be received with high sensitivity and can be imaged with high clarity.

As described with reference to diagrams such as FIG. 13, the light L after passing through the imaging lens 4 may be incident on the prism 6, the normal visible light VL1 the visible special light VL2, and the special light FL may have individually different optical path lengths from the incidence of the light L on the prism 6 to image forming, the visible light imaging sensor 7 may be directly or indirectly fixed to the prism 6 so as to be located at the image forming position of the visible light VL, the special light imaging sensor 9 may be directly or indirectly fixed to the prism 6 so as to be located at the image forming position of the special light FL, and the visible special light imaging sensor 12 may be directly or indirectly fixed to the prism 6 so as to be located at the image forming position of the visible special light VL2. With this configuration, the special light imaging sensor 9 and the visible special light imaging sensor 12 can achieve focusing only by focusing the visible light imaging sensor 7, facilitating imaging of the normal visible light VL1 with high resolution and imaging of the special light FL and the visible special light VL2 with high clarity.

As described with reference to diagrams such as FIG. 13, the imaging device 1 may include the band pass filter 81 (first band pass filter) that is provided between the prism 6 and the special light imaging sensor 9 and allows passage of the special light FL; and the band pass filter 82 (second band pass filter) that is provided between the prism 6 and the visible special light imaging sensor 12 and allows passage of the visible special light VL2. The band pass filter 81 may have an optical axis direction length designed in accordance with an optical path length difference between the optical path lengths of the visible light VL and the special light FL so as to position the special light imaging sensor 9 at the image forming position of the special light FL. The band pass filter 82 may have an optical axis direction length designed in accordance with an optical path length difference between the optical path lengths of the visible light VL and the visible special light VL2 so as to position the visible special light imaging sensor 12 at the image forming position of the visible special light VL2. The prism 6 may have an optical axis direction length designed in accordance with the above-described optical path length differences between each of the optical path lengths of the normal visible light VL1, special light FL, and the visible special light VL2 so as to position the visible light imaging sensor 7 at the image forming position of the visible light VL, position the special light imaging sensor 9 at the image forming position of the special light FL, and position the visible special light imaging sensor 12 at the image forming position of the visible special light VL2. The imaging device 1 may include the void G positioned between the prism 6 and at least one of the visible light imaging sensor 7, the special light imaging sensor 9, and the visible special light imaging sensor 12. The void G may have an optical axis direction length designed in accordance with an optical path length difference between the optical path lengths of the normal visible light VL1, the special light FL, and the visible special light VL2 so as to position the visible light imaging sensor 7 at the image forming position of the visible light VL, position the special light imaging sensor 9 at the image forming position of the special light FL, and position the visible special light imaging sensor 12 at the image forming position of the visible special light VL2. The visible light imaging sensor 7, the special light imaging sensor 9, and the visible special light imaging sensor 12 can be positioned in this manner, for example.

The medical observation system (for example, the endoscope system 5000 and the microscopic surgery system 5300) described with reference to FIGS. 16 to 18 is also one of the disclosed technologies. The medical observation system includes the imaging device 1 that images a surgical site. The imaging device 1 is as described above. In medical observation, even when the visible light VL includes the normal visible light VL1 and the visible special light VL2, and both of these beams of light are to be observed, the visible special light VL2 can be imaged with high clarity.

Note that the effects described in the present disclosure are merely examples and are not limited to the disclosed contents. There may be other effects.

The embodiments of the present disclosure have been described above. However, the technical scope of the present disclosure is not limited to the above-described embodiments, and various modifications can be made without departing from the scope of the present disclosure. Moreover, it is allowable to combine the components across different embodiments and modifications as appropriate.

Note that the present technology can also have the following configurations.
(1) An imaging device comprising:
   a prism configured to separate visible light and special light from each other, the visible light and the special light included in light from an observation target;
   a visible light imaging sensor configured to image the visible light separated by the prism; and
   a special light imaging sensor configured to image the special light separated by the prism, wherein
   a ratio of spectral transmittance of the visible light toward the special light imaging sensor to spectral transmittance of the visible light toward the visible light imaging sensor in the prism is 0.25 or more and 4 or less, and
   spectral transmittance of the special light toward the special light imaging sensor in the prism is greater than 90%.
(2) The imaging device according to (1), further comprising
   a depolarizer provided between the observation target and the prism.
(3) The imaging device according to (1) or (2), wherein
   the visible light includes normal visible light and visible special light.
(4) The imaging device according to (3), further comprising
   a band pass filter provided between the prism and the special light imaging sensor and configured to pass the visible special light and the special light, wherein
   the special light imaging sensor also images the visible special light.
(5) The imaging device according to any one of (1) to (4), wherein
   a ratio of a pixel interval of the special light imaging sensor to a pixel interval of the visible light imaging sensor is 1 or more and less than 5.
(6) The imaging device according to any one of (1) to (5), wherein
   a ratio of an effective pixel area diagonal length of the special light imaging sensor to an effective pixel area diagonal length of the visible light imaging sensor is greater than 0.7 and less than 1.4.
(7) The imaging device according to any one of (1) to (6), wherein
   light after passing through an imaging lens is incident on the prism,
   the visible light and the special light have individually different optical path lengths from the incidence of the light on the prism to image forming,
   the visible light imaging sensor is directly or indirectly fixed to the prism so as to be located at an image forming position of the visible light, and
   the special light imaging sensor is directly or indirectly fixed to the prism so as to be located at an image forming position of the special light.
(8) The imaging device according to (7), wherein
   the visible light includes normal visible light and visible special light,
   the imaging device comprises a band pass filter provided between the prism and the special light imaging sensor and configured to transmit the visible special light and the special light, and
   the band pass filter has an optical axis direction length designed in accordance with an optical path length difference between the optical path lengths of the visible light and the special light so as to position the special light imaging sensor at the image forming position of the special light.
(9) The imaging device according to (7) or (8), wherein
   the prism has an optical axis direction length designed in accordance with an optical path length difference between the optical path lengths of the visible light and the special light so as to position the visible light imaging sensor at the image forming position of the visible light and position the special light imaging sensor at the image forming position of the special light.
(10) The imaging device according to any one of (7) to (9), further comprising
   a void positioned between the prism and at least one of the visible light imaging sensor and the special light imaging sensor, wherein
   the void has an optical axis direction length designed in accordance with an optical path length difference between the optical path lengths of the visible light and the special light so as to position the visible light imaging sensor at the image forming position of the visible light and position the special light imaging sensor at the image forming position of the special light.
(11) The imaging device according to any one of (1) to (10), further comprising:
   a light source configured to irradiate the observation target with white light and excitation light at different timings from each other; and
   a signal processing section configured to process an imaging signal from the visible light imaging sensor, wherein
   the visible light includes normal visible light and visible special light,
   the normal visible light includes light obtained by reflecting the white light from the light source on the observation target,
   the visible special light includes light emitted by the observation target excited by the irradiation of the excitation light from the light source, and
   the signal processing section processes the imaging signal when the observation target is irradiated with the white light as a normal visible light image signal, and processes the imaging signal when the observation target is irradiated with the excitation light as a visible special light image signal.
(12) An imaging device comprising:
   a prism configured to separate visible light and special light from each other, the visible light and the special light included in light from an observation target;
   a visible light imaging sensor configured to image the visible light separated by the prism;
   a special light imaging sensor configured to image the special light separated by the prism; and
   a visible special light imaging sensor configured to image visible special light included in the visible light separated by the prism, wherein
   a ratio of spectral transmittance of the visible light toward the visible special light imaging sensor to spectral transmittance of the visible light toward the visible light imaging sensor in the prism is greater than 0.2 and less than 5, and
   spectral transmittance of the special light toward the special light imaging sensor in the prism is greater than 90%.
(13) The imaging device according to (12), further comprising at least one of:
   a depolarizer provided between the observation target and the prism; and
   a band pass filter provided between the prism and the visible special light imaging sensor and configured to allow passage of the visible special light.
(14) The imaging device according to (12) or (13), wherein
   a ratio of a pixel interval of the visible special light imaging sensor to a pixel interval of the visible light imaging sensor, and a ratio of a pixel interval of the special light imaging sensor to the pixel interval of the visible light imaging sensor, are both 1 or more and less than 5.
(15) The imaging device according to any one of (12) to (14), wherein
   a ratio of an effective pixel area diagonal length of the visible special light imaging sensor to an effective pixel area diagonal length of the visible light imaging sensor, and a ratio of an effective pixel area diagonal length of the special light imaging sensor to the effective pixel area diagonal length of the visible light imaging sensor, are both greater than 0.7 and less than 1.4.
(16) The imaging device according to any one of (12) to (15), wherein
   the visible light includes normal visible light and the visible special light,
   light after passing through an imaging lens is incident on the prism,
   the normal visible light, the special light, and the visible special light have individually different optical path lengths from the incidence of the light on the prism to image forming,
   the visible light imaging sensor is directly or indirectly fixed to the prism so as to be located at an image forming position of the visible light,
   the special light imaging sensor is directly or indirectly fixed to the prism so as to be located at an image forming position of the special light, and
   the visible special light imaging sensor is directly or indirectly fixed to the prism so as to be located at an image forming position of the visible special light.
(17) The imaging device according to (16), further comprising:
   a first band pass filter provided between the prism and the special light imaging sensor and configured to allow passage of the special light; and
   a second band pass filter provided between the prism and the visible special light imaging sensor and configured to allow passage of the visible special light, wherein
   the first band pass filter has an optical axis direction length designed in accordance with an optical path length difference between the optical path lengths of the visible light and the special light so as to position the special light imaging sensor at the image forming position of the special light, and
   the second band pass filter has an optical axis direction length designed in accordance with an optical path length difference between the optical path lengths of the visible light and the visible special light so as to position the visible special light imaging sensor at the image forming position of the visible special light.
(18) The imaging device according to (16) or (17), wherein
   the prism has an optical axis direction length designed in accordance with an optical path length difference between the optical path lengths of the normal visible light, the special light, and the visible special light so as to position the visible light imaging sensor at the image forming position of the visible light, position the special light imaging sensor at the image forming position of the special light, and position the visible special light imaging sensor at the image forming position of the visible special light.
(19) The imaging device according to any one of (16) to (18), further comprising
   a void positioned between the prism and at least one of the visible light imaging sensor, the special light imaging sensor, and the visible special light imaging sensor, wherein
   the void has an optical axis direction length designed in accordance with an optical path length difference between the optical path lengths of the normal visible light, the special light, and the visible special light so as to position the visible light imaging sensor at the image forming position of the visible light, position the special light imaging sensor at the image forming position of the special light, and position the visible special light imaging sensor at the image forming position of the visible special light.
(20) A medical observation system comprising
   an imaging device configured to image a surgical site, wherein
   the imaging device includes:
   a prism configured to separate visible light and special light from each other, the visible light and the special light included in light from the surgical site;
   a visible light imaging sensor configured to image the visible light separated by the prism; and
   a special light imaging sensor configured to image the special light separated by the prism,
   a ratio of spectral transmittance of the visible light toward the special light imaging sensor to spectral transmittance of the visible light toward the visible light imaging sensor in the prism is 0.25 or more and 4 or less, and
   spectral transmittance of the special light toward the special light imaging sensor from the prism is greater than 90%.

### REFERENCE SIGNS LIST

1 IMAGING DEVICE
2 LIGHT SOURCE
21 WHITE LIGHT SOURCE
22 VISIBLE LIGHT EXCITATION LIGHT SOURCE
23 INVISIBLE LIGHT EXCITATION LIGHT SOURCE
3 OPTICAL DIAGNOSIS ASSEMBLY
4 IMAGING LENS
4a INCOMING SURFACE
4b OUTGOING SURFACE
5 OPTICAL ELEMENT (DEPOLARIZER, EXCITATION LIGHT CUT FILTER)
5a INCOMING SURFACE
5b OUTGOING SURFACE
6 PRISM
6a INCOMING SURFACE
6b1 OUTGOING SURFACE
6b2 OUTGOING SURFACE
6b11 OUTGOING SURFACE
6b12 OUTGOING SURFACE
61 BLOCK
62 BLOCK
621 BLOCK
622 BLOCK
65 DICHROIC FILM
66 DICHROIC FILM
7 VISIBLE LIGHT IMAGING SENSOR
7a LIGHT RECEIVING SURFACE
7p PIXEL
7a1 PIXEL AREA
7a2 EFFECTIVE PIXEL AREA
8 BAND PASS FILTER
8a INCOMING SURFACE
8b OUTGOING SURFACE
81 BAND PASS FILTER
82 BAND PASS FILTER
9 SPECIAL LIGHT IMAGING SENSOR
9a LIGHT RECEIVING SURFACE
9p PIXEL
9a1 PIXEL AREA
9a2 EFFECTIVE PIXEL AREA
10 HOLDING MEMBER
101 SUBSTRATE
102 HOLDING ROD
102a ONE END
102b OTHER END
11 PROCESSING UNIT
111 LIGHT SOURCE CONTROLLER
112 IMAGING CONTROLLER
113 SIGNAL PROCESSING SECTION
5000 ENDOSCOPE SYSTEM (MEDICAL OBSERVATION SYSTEM)
5300 MICROSCOPIC SURGERY SYSTEM (MEDICAL OBSERVATION SYSTEM)
D1 EFFECTIVE PIXEL AREA DIAGONAL LENGTH
D2 EFFECTIVE PIXEL AREA DIAGONAL LENGTH
D3 EFFECTIVE PIXEL AREA DIAGONAL LENGTH
D4 EFFECTIVE PIXEL AREA DIAGONAL LENGTH
D5 EFFECTIVE PIXEL AREA DIAGONAL LENGTH
G VOID
P1 PIXEL INTERVAL
P2 PIXEL INTERVAL
P3 PIXEL INTERVAL
P4 PIXEL INTERVAL
P5 PIXEL INTERVAL
S OBSERVATION TARGET
L LIGHT
SL LIGHT
VL VISIBLE LIGHT
VL1 NORMAL VISIBLE LIGHT
VL2 VISIBLE SPECIAL LIGHT
WL WHITE LIGHT
EL1 EXCITATION LIGHT
EL2 EXCITATION LIGHT
W1 PIXEL SIZE
W2 PIXEL SIZE

1. An imaging device comprising:
a prism configured to separate visible light and special light from each other, the visible light and the special light included in light from an observation target;
a visible light imaging sensor configured to image the visible light separated by the prism; and
a special light imaging sensor configured to image the special light separated by the prism, wherein
a ratio of spectral transmittance of the visible light toward the special light imaging sensor to spectral transmittance of the visible light toward the visible light imaging sensor in the prism is 0.25 or more and 4 or less, and
spectral transmittance of the special light toward the special light imaging sensor in the prism is greater than 90%.

2. The imaging device according to claim 1, further comprising
a depolarizer provided between the observation target and the prism.

3. The imaging device according to claim 1, wherein
the visible light includes normal visible light and visible special light.

4. The imaging device according to claim 3, further comprising
a band pass filter provided between the prism and the special light imaging sensor and configured to pass the visible special light and the special light, wherein
the special light imaging sensor also images the visible special light.

5. The imaging device according to claim 1, wherein
a ratio of a pixel interval of the special light imaging sensor to a pixel interval of the visible light imaging sensor is 1 or more and less than 5.

6. The imaging device according to claim 1, wherein
a ratio of an effective pixel area diagonal length of the special light imaging sensor to an effective pixel area diagonal length of the visible light imaging sensor is greater than 0.7 and less than 1.4.

7. The imaging device according to claim 1, wherein
light after passing through an imaging lens is incident on the prism,
the visible light and the special light have individually different optical path lengths from the incidence of the light on the prism to image forming,
the visible light imaging sensor is directly or indirectly fixed to the prism so as to be located at an image forming position of the visible light, and
the special light imaging sensor is directly or indirectly fixed to the prism so as to be located at an image forming position of the special light.

8. The imaging device according to claim 7, wherein
the visible light includes normal visible light and visible special light,
the imaging device comprises a band pass filter provided between the prism and the special light imaging sensor and configured to transmit the visible special light and the special light, and
the band pass filter has an optical axis direction length designed in accordance with an optical path length difference between the optical path lengths of the visible light and the special light so as to position the special light imaging sensor at the image forming position of the special light.

9. The imaging device according to claim 7, wherein
the prism has an optical axis direction length designed in accordance with an optical path length difference between the optical path lengths of the visible light and the special light so as to position the visible light imaging sensor at the image forming position of the visible light and position the special light imaging sensor at the image forming position of the special light.

10. The imaging device according to claim 7, further comprising
a void positioned between the prism and at least one of the visible light imaging sensor and the special light imaging sensor, wherein
the void has an optical axis direction length designed in accordance with an optical path length difference between the optical path lengths of the visible light and the special light so as to position the visible light imaging sensor at the image forming position of the visible light and position the special light imaging sensor at the image forming position of the special light.

11. The imaging device according to claim 1, further comprising:
a light source configured to irradiate the observation target with white light and excitation light at different timings from each other; and
a signal processing section configured to process an imaging signal from the visible light imaging sensor, wherein
the visible light includes normal visible light and visible special light,
the normal visible light includes light obtained by reflecting the white light from the light source on the observation target,
the visible special light includes light emitted by the observation target excited by the irradiation of the excitation light from the light source, and
the signal processing section processes the imaging signal when the observation target is irradiated with the white light as a normal visible light image signal, and processes the imaging signal when the observation target is irradiated with the excitation light as a visible special light image signal.

12. An imaging device comprising:
a prism configured to separate visible light and special light from each other, the visible light and the special light included in light from an observation target;
a visible light imaging sensor configured to image the visible light separated by the prism;
a special light imaging sensor configured to image the special light separated by the prism; and
a visible special light imaging sensor configured to image visible special light included in the visible light separated by the prism, wherein
a ratio of spectral transmittance of the visible light toward the visible special light imaging sensor to spectral transmittance of the visible light toward the visible light imaging sensor in the prism is greater than 0.2 and less than 5, and
spectral transmittance of the special light toward the special light imaging sensor in the prism is greater than 90%.

13. The imaging device according to claim 12, further comprising at least one of:
a depolarizer provided between the observation target and the prism; and
a band pass filter provided between the prism and the visible special light imaging sensor and configured to allow passage of the visible special light.

14. The imaging device according to claim 12, wherein
a ratio of a pixel interval of the visible special light imaging sensor to a pixel interval of the visible light imaging sensor, and a ratio of a pixel interval of the special light imaging sensor to the pixel interval of the visible light imaging sensor, are both 1 or more and less than 5.

15. The imaging device according to claim 12, wherein
a ratio of an effective pixel area diagonal length of the visible special light imaging sensor to an effective pixel area diagonal length of the visible light imaging sensor, and a ratio of an effective pixel area diagonal length of the special light imaging sensor to the effective pixel area diagonal length of the visible light imaging sensor, are both greater than 0.7 and less than 1.4.

16. The imaging device according to claim 12, wherein
the visible light includes normal visible light and the visible special light,
light after passing through an imaging lens is incident on the prism,
the normal visible light, the special light, and the visible special light have individually different optical path lengths from the incidence of the light on the prism to image forming,
the visible light imaging sensor is directly or indirectly fixed to the prism so as to be located at an image forming position of the visible light,
the special light imaging sensor is directly or indirectly fixed to the prism so as to be located at an image forming position of the special light, and
the visible special light imaging sensor is directly or indirectly fixed to the prism so as to be located at an image forming position of the visible special light.

17. The imaging device according to claim 16, further comprising:
a first band pass filter provided between the prism and the special light imaging sensor and configured to allow passage of the special light; and
a second band pass filter provided between the prism and the visible special light imaging sensor and configured to allow passage of the visible special light, wherein
the first band pass filter has an optical axis direction length designed in accordance with an optical path length difference between the optical path lengths of the visible light and the special light so as to position the special light imaging sensor at the image forming position of the special light, and
the second band pass filter has an optical axis direction length designed in accordance with an optical path length difference between the optical path lengths of the visible light and the visible special light so as to position the visible special light imaging sensor at the image forming position of the visible special light.

18. The imaging device according to claim 16, wherein
the prism has an optical axis direction length designed in accordance with an optical path length difference between the optical path lengths of the normal visible light, the special light, and the visible special light so as to position the visible light imaging sensor at the image forming position of the visible light, position the special light imaging sensor at the image forming position of the special light, and position the visible special light imaging sensor at the image forming position of the visible special light.

19. The imaging device according to claim 16, further comprising
a void positioned between the prism and at least one of the visible light imaging sensor, the special light imaging sensor, and the visible special light imaging sensor, wherein
the void has an optical axis direction length designed in accordance with an optical path length difference between the optical path lengths of the normal visible light, the special light, and the visible special light so as to position the visible light imaging sensor at the image forming position of the visible light, position the special light imaging sensor at the image forming position of the special light, and position the visible special light imaging sensor at the image forming position of the visible special light.

20. A medical observation system comprising
an imaging device configured to image a surgical site, wherein
the imaging device includes:
a prism configured to separate visible light and special light from each other, the visible light and the special light included in light from the surgical site;
a visible light imaging sensor configured to image the visible light separated by the prism; and
a special light imaging sensor configured to image the special light separated by the prism,
a ratio of spectral transmittance of the visible light toward the special light imaging sensor to spectral transmittance of the visible light toward the visible light imaging sensor in the prism is 0.25 or more and 4 or less, and
spectral transmittance of the special light toward the special light imaging sensor from the prism is greater than 90%.
